(19) United States
(12) Patent Application Publication
(10) Pub. No.: US 2008/0281167 A1
(43) Pub. Date: Nov. 13, 2008

(54) DIAGNOSTIC INSTRUMENT WORKSTATION

(75) Inventors: Peter H. Soderberg, Skaneateles, NY (US); David G. Perkins, Tully, NY (US); Bonita L. Labosky, Skaneateles, NY (US)

Correspondence Address: Hiscock & Barclay, LLP, One Park Place, 300 South State Street, Syracuse, NY, 13202-2078, US

(73) Assignee: Welch Allyn, Inc., Skaneateles Falls, NY (US)

(21) Appl. No.: 12/125,107

(22) Filed: May 22, 2008

Related U.S. Application Data
(62) Division of application No. 10/643,487, filed on Aug. 19, 2003.
(60) Provisional application No. 60/404,601, filed on Aug. 20, 2002.

(51) Int. Cl. A61B 5/00

(52) U.S. Cl. 600/300

(57) ABSTRACT

An integrated medical workstation for use in patient clinical encounters includes an input device such as a bar code scanner that is interconnected to a computing device. At least one device capable of obtaining at least one physiological parameter is either attached directly to the workstation or is in communication therewith. Preferably, the input scanning device controls at least substantial overall operation of the medical workstation that can be placed, for example, into a network.

### CROSS-REFERENCE TO RELATED APPLICATIONS

This patent application is a divisional application of U.S. Ser. No. 10/643,487, filed Aug. 19, 2003, which claims priority under 35 USC §119(e) based upon the following commonly owned provisional patent application: U.S. Ser. N0. 60/404,601, filed Aug. 20, 2002, the entire contents of which are incorporated herein by reference.

### FIELD OF THE INVENTION

This invention relates generally to the field of medicine and in particular to an integrated medical diagnostic platform or workstation that can be used for clinical encounters between patients and physicians, as well as other health care professionals.

### BACKGROUND OF THE INVENTION

The staff of a medical/surgical floor of a typical hospital is under a number of considerable pressures. Adding to these is the pervasive nursing shortage which has translated into a higher nurse to patient ratio, with longer hours and increased overtime. As a result, errors due to oversight and the like are likely to increase. Formerly, patient vital sign data was taken by the registered nurse (RN), but now these readings are often taken numerous (as many as six or more) times a day by nursing aides, who cover more patients and have less clinical knowledge. In addition and in an effort to ease the strain, hospitals utilize more “travelers”—that is, temporary contract nurses, wherein nurses often will float between units. Therefore, users are transitory and must learn new internal procedures very quickly, exacerbating the above problems.

Currently, nursing aides use a cart having a number of patient diagnostic apparatus including separate automated blood pressure, thermometry, and pulse oximetry devices to take patient vital signs over a length of stay. On average, a nursing aide will take about six readings per day over an average hospital stay of about five days. Typically the above devices are not integrated on the cart, but are arranged in a piecemeal fashion, though there are known integrated vital sign monitoring devices, such as those manufactured by Welch Allyn Inc., of Skaneateles, N.Y., among others.

Vital sign readings, when taken, are usually written down on a work sheet or often on scraps of paper. At the end of rounds, these readings are copied onto the patient's chart on a “vitals” sheet. If anomalous readings are noticed, the RN is notified. Otherwise, the RN is not consulted and often will not or may not get the chance to review the readings which have been taken.

Upon examination, and if the vital sign readings are suspect in any way, the RN will often send the aide back to the patient and request that another reading(s) be taken. In the meantime, even if a significant change in the patient's vital signs has been detected, time has been wasted and therefore lost. It is possible in the current manner of testing, that many vital sign variations are not caught or otherwise detected or noticed until the patient's condition has changed significantly.

Though the problems are arguably less involved, there are similar generalized needs in the field required for physician's or other healthcare provider's offices to be able to better conduct and document patient clinical encounters more efficiently.

### SUMMARY OF THE INVENTION

It is therefore a primary object of the present invention to overcome the above-noted deficiencies of the prior art.

It is another primary object of the present invention to improve he conduction of patient clinical encounters, whether in a doctor's office and/or in the hospital environment.

It is another primary object of the present invention to be able to ease the strain presently found in the hospital/clinical environment by developing an integrated medical diagnostic workstation that provides simple, efficient and improved operation for both the patient and the user.

It is another primary object of the present invention to be able to better control and efficiently track inventory and supply aspects relating to instruments and patient-related items, including but not limited to disposable and nondisposables and medications, that are utilized during the course of a hospital visit or during a clinical encounter.

It is yet another primary object of the present invention to be able to substantially reduce or eliminate billing errors made during hospital visits and/or relating to clinical encounters by providing a better tracking system for inventory, medications, and procedures.

Therefore and according to a preferred aspect of the invention, there is provided an integrated instrument workstation for use in a patient encounter, the apparatus comprising at least one medical instrument, an input device having means for reading machine-readable information, and a computing device connected to said at least one medical instrument and said input device. Preferably, the input device is a scanning apparatus, such as a bar-code scanner, that is capable of identifying and decoding machine-coded information wherein the workstation includes a set of machine-coded and executable instructions/commands that are selectively executed by use of the scanner, wherein use of the scanning device enables substantial operation of the diagnostic workstation, including the taking of patient vital signs, and/or other aspects of the workstation.

According to another preferred aspect of the invention, image data can be selectively captured by means of a miniature imaging device that is interconnected to the computing device and the input device. Image data can be selectively captured and stored into the memory of the computing device, for example, using the scanning device, and/or printed into a suitable format for use by the physician as part of a patient chart or for updating patient information. In addition, physiological information, such as vital sign information including but not limited to blood pressure, pulse rate, SPO₂ and other patient-related data can also be collected with the results being selectively stored for data logging and/or trending as needed or for printing as a summary or other report.

According to another preferred embodiment, the image capture means can include the scanner which, if an imager-based scanner, can perform as an imager to capture images as well as permit encoded patient, physician, device and/or medication/treatment information to be scanned and stored as needed. According to a preferred embodiment, a captured image can be analyzed in order to detect the presence of machine-coded information which when detected is then decoded automatically.

According to yet another preferred embodiment, the entire operation of the herein described integrated workstation can be performed entirely or in large part by using the input device, wherein the receipt and scanning and decoding of machine-coded information in combination with software contained within the computing device, provides efficient and repeatable operation of the workstation. In such apparatus, the operation can be greatly simplified and made more efficient while errors can be significantly reduced at the same time. Alternately, the workstation can be operated manually; that is, through use of a keyboard and/or the at least one medical instrument (e.g., a vital signs collector, infusion pump, etc.,), or through use of discrete control buttons disposed on a console of the workstation, as needed.

To prevent the workstation and/or patient-related data from being used without proper authorization and to comply with government regulations such as HIPAA, encryption means, such as finger print verification devices and/or other biometric authorization access devices, can be easily added or incorporated for use with the workstation to prevent unauthorized users from being able to operate same.

The storage of patient-specific information permits customized usage, for example, in conjunction with a pressure control assembly of a sphygmomanometer to automatically control the inflation of the blood pressure sleeve, depending on whether the patient, for example, is hypotensive or hypertensive.

In addition, the workstation can be configured to detect other patient-related data, such as fluid input and outputs information. Preferably, this detection can be done automatically using the scanning device and machine coded fluid identification tags on a fluid container, such as a patient drinking cup and/or bottle.

According to yet another aspect of the invention, the workstation can include an interrogation device that transmits a locator signal in order to identify instrument and other articles that include a passive locator tag. The tag emits a response signal using RF or other means to the workstation, permitting identification of certain equipment in an examination room, for example.

The workstation can wirelessly communicate using Bluetooth, WiFi, or other known protocol with at least one medical diagnostic instrument, including that which is remote from the workstation itself, but within an examination room, such as a digital scale, a vital signs collector or an infusion pump. By way of this communication access, that is preferably bi-directional, the at least one medical diagnostic instrument can be operated with the results/data being transmitted to the workstation for storage and/or data archiving.

The workstation can also be added to a hospital network, in which the workstation can be either hard wired or wirelessly connected thereto, as a single unit, or preferably in conjunction with a plurality of workstations. As such, data can be uploaded from any workstation for central storage into a central server.

Other data in addition to image data can be conveyed, for example, voice (audio) data can be stored using the workstation during a patient encounter. Moreover, the voice data can also be remotely transmitted, for example, to an RN or other caregiver, as needed.

An advantage of the present invention is that patient/physician clinical encounters, whether in a physician's office or a hospital setting, are now more comprehensive and efficient as well as cost effective.

Still another advantage is that a workstation can be provided which carries all or substantially all of the inventory required for a clinical encounter, including spare equipment such as varying sized blood pressure cuffs, EKG electrodes and the like, thereby saving time in having to locate such items separately.

Yet another advantage is that the workstation permits customization to a specific patient(s), for example, to provide an alert automatically when a patient's vital signs are not within a prescribed range or envelope of readings through data trending and datalogging.

Still another advantage is that the use of a bar code scanner or other similar device permits the workstation to scan multiple items, not only instructions for the operation of the workstation but also patient-related items such as disposable probes, and similar items. The workstation can also be used to inventory items used on the cart through a similar scanning procedure. Moreover, patient medications and pharmaceuticals can also be tracked and verified in the same way with the results being stored into the memory of the computing device.

The herein described workstation through its automation permits a patient to receive adequate care without necessarily requiring professional trained personnel (RNs), freeing the latter to handle more urgent matters, unless so required. In addition, the workstation facilitates training for newer aides and other health providers, again due to its simplified operation and automated features.

Additional equipment can be easily added based on the workstation's architecture. For example, a wireless link can be added which can scan for RE or other forms of identifiable tags to determine the location of other equipment in a hospital or examination room and/or a global positioning system (GPS) to locate the position of the workstation.

These and other objects, features and advantages will be apparent from the following Detailed Description which should be read in conjunction with the accompanying drawings.

### BRIEF DESCRIPTION OF THE DRAWINGS

FIG. 1 is a front perspective view of a medical diagnostic workstation in accordance with a first embodiment of the present invention;

FIG. 2 is an enlarged view of the top portion of the medical diagnostic workstation of FIG. 1;

FIG. 3 is a rear perspective view of the medical diagnostic workstation of FIGS. 1 and 2;

FIG. 4 is a partial enlarged view of the top portion of the medical diagnostic workstation of FIG. 3;

FIG. 5 is a partial enlarged view of the top of the medical diagnostic workstation depicting a wireless connection between the computing device and the vital sign collector;

FIG. 5(a) is a schematic diagram of the medical diagnostic workstation of FIGS. 1-5;

FIG. 6 is a front view of a medical diagnostic workstation in accordance with a second embodiment of the present invention;

FIG. 7 is a side view of the medical diagnostic workstation of FIG. 6;

FIG. 8 is a top view of the medical diagnostic workstation of FIGS. 6 and 7;

FIG. 9 is a side perspective view of the medical diagnostic workstation of FIGS. 6-8;

FIG. 10 is a full front view of the medical diagnostic workstation of FIG. 9;

FIG. 11 is a partial front view of the medical diagnostic workstation of FIGS. 9 and 10;

FIG. 12 is another partial side perspective view of the medical diagnostic workstation of FIGS. 9-11, depicting a miniature video camera for use therewith and its receiving cradle;

FIG. 13 is an enlarged view of the miniature video camera and interface of the medical diagnostic workstation of FIGS. 9-12;

FIG. 14 is a partial side perspective view of the medical diagnostic workstation of FIGS. 9-13, depicting a wireless link between the vital signs collector and the computing device;

FIG. 15 is a front view of a printer and a wireless link used in conjunction with the medical diagnostic workstation of FIGS. 9-14;

FIG. 16 is a partial rear perspective view of the medical diagnostic workstation of FIGS. 9-15, illustrating the receiving cradle and a miniature video camera;

FIG. 17 is a partial front view of another miniature video camera for use with the medical diagnostic workstation of the present invention according to a preferred embodiment;

FIG. 17(a) is a rear view of the miniature video camera of FIG. 17;

FIG. 18 depicts a miniature video camera made in accordance with the present invention and having an integral video display;

FIG. 19 depicts a wall mounted diagnostic workstation for the miniature video camera of FIG. 18;

FIG. 20 is a typical patient summary data sheet format which is obtainable with the medical diagnostic workstation of FIGS. 9-15;

FIG. 21 is a functional block diagram for a medical diagnostic workstation made in accordance with a third embodiment of the invention;

FIG. 22 is a front perspective view of an integrated medical diagnostic workstation according to a fourth embodiment of the present invention;

FIG. 23 is a partial front view of the medical diagnostic workstation of FIG. 22;

FIG. 24 is a partial side view of the medical diagnostic workstation of FIGS. 22 and 23;

FIG. 25 is a partial front perspective view, angled approximately 45 degrees, of the medical diagnostic workstation of FIGS. 22-24;

FIG. 26 is another partial front perspective view of the medical diagnostic workstation of FIGS. 22-25;

FIG. 27 is a side perspective view of a medical diagnostic workstation made in accordance with a fifth embodiment of the invention and in an unused position;

FIG. 28 is a rear perspective view of the medical diagnostic workstation of FIG. 27;

FIG. 29 is a perspective view taken from the opposite side, relative to FIG. 27, of the medical diagnostic workstation of FIGS. 27 and 28;

FIG. 30 is the side perspective view of FIG. 27 illustrating the medical diagnostic workstation in a use position;

FIG. 31 is the rear perspective view of the medical diagnostic workstation of FIG. 28, with the rear panel removed to illustrate a preferred position of a contained vital signs collector;

FIG. 32 is a sample display output of a patient record of the medical diagnostic workstation of FIGS. 27-31;

FIGS. 33-43 present front perspective renderings of sixth and alternate embodiments of medical diagnostic workstations that are also made in accordance with the present invention;

FIG. 44 is a front perspective view of a wall mounted medical diagnostic workstation made in accordance with another preferred embodiment of the invention;

FIG. 45 is a front perspective view of a diagnostic workstation in accordance with a seventh embodiment of the present invention;

FIG. 46 is a pictorial representation, partially diagrammatic, illustrating the diagnostic workstation of FIG. 45 as used in a clinical environment;

FIG. 47 is a front view of a graphical user interface of the diagnostic workstation of FIGS. 45 and 46; and

FIGS. 48-50 represent diagrams of single and multiple configurations involving the diagnostic workstations of FIGS. 47-49.

### DETAILED DESCRIPTION

The following discussion relates to several preferred embodiments of an integrated medical instrument workstation according to the present invention. It will be readily apparent to those of sufficient skill in the field that numerous modifications and variations are possible within the intended scope of the invention. In addition, several terms are used in this description in order to provide a suitable frame of reference with regard to the accompanying drawings. These terms are not intended, however, to be limiting the invention, except where so expressly stated.

A number of other terms are also used throughout the discussion which should bear additional explanation and clarification before further discussion of the embodiments of the present invention is added.

The term “medical instrument” is used to include any device that can be used in conjunction with a patient for purposes of documentation, diagnosis, treatment or therapy during a patient encounter.

The term “computing device” as used herein refers to any form of processing engine, such as a portable laptop computer or personal data assistant (e.g., a PDA), etc., but should not be limited in structure to hardware having a defined housing. That is, a suitable I/O integrated circuit board linked, for example, to other circuitry and having solid state memory can be conveniently utilized herein according to the invention.

The term “vital signs collector” or “vital signs monitor” as used herein refers to any device or apparatus that is capable of collecting a varied number of physiological parameter/patient vital sign readings, including but not limited to blood pressure, EKG, pulse oximetry, body temperature, and pulse rate.

The term “cart” as used herein refers to a movable supporting structure that supports a number of discrete components.

The term “support or supporting structure” refers to any form of frame or other support capable of retaining a number of discrete medical and other components.

The term “machine-readable information” or “machine-readable code” as used herein refers to information which can be read (e.g., scanned) and interpreted by a machine. This can include, for example, one dimensional (1D) and two dimensional (2D) bar-code symbologies, as well as optical character recognition (OCR) symbols. This term can also refer more simply to identification of any other machine perceivable information, such as color, or physical parameters such as sound and the like. For example, the above definition can apply to a passive radio frequency (RF) tag that can be used to identify the location of an article or device that can be located by means of an interrogatory device.

The term “wireless” refers to any communication technique which does not require a hardwired connection. Such radio frequency protocols as Bluetooth, WiFi (802.11(b)) Zigbee, frequency hopping, and 802.11(a) and 802.11 (g) are included in this definition as well as infrared (IR), optical, and other radio frequency (RF) techniques, among others.

### FIRST EMBODIMENT

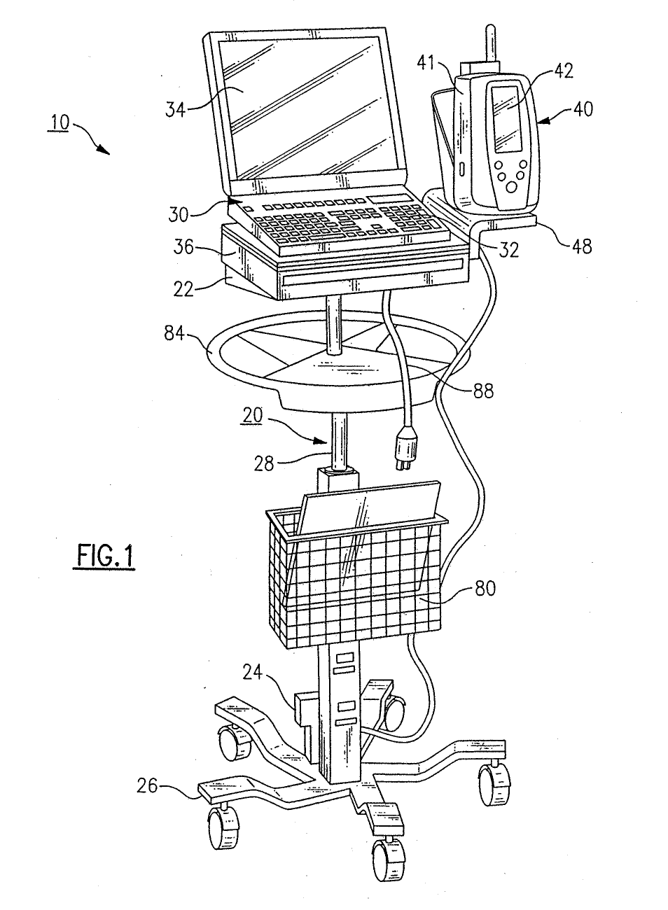

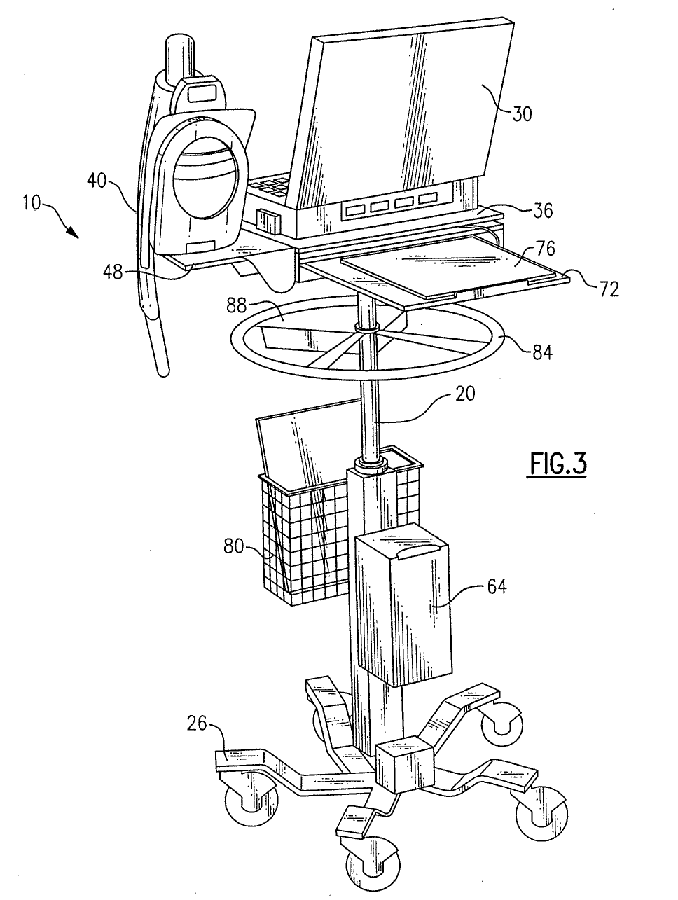

Referring to FIGS. 1-5(a), there is herein described a medical diagnostic workstation 10 that is manufactured in accordance with a first working embodiment of the present invention. The medical diagnostic workstation 10 includes a movable supporting structure 20 that supports a number of discrete components to form an integrated assembly. Among these components are a computing device 30 and a vital signs collector 40, each of which are retained on an upper portion 22 of the supporting structure 20.

The computing device 30, according to this embodiment, is a portable laptop computer that includes a keyboard 32 as well as a thin film transistor (TFT) or other form of LED display 34. As noted above and as will be critical to an understanding of the present invention, the term “computing device” as designated herein is intended to encompass computer hardware, such as laptops and personal data assistants (PDAs), but overall it is better to consider the computing device in terms of its processing capability only. That is to say, the term is intended to read more upon the internal functionality of the computing device in the main, meaning I/O integrated circuit boards and/or literally any form of processing engine and adequate solid state storage memory, without specifically limiting the definition to any concrete form of supporting structure (e.g., a discrete housing).

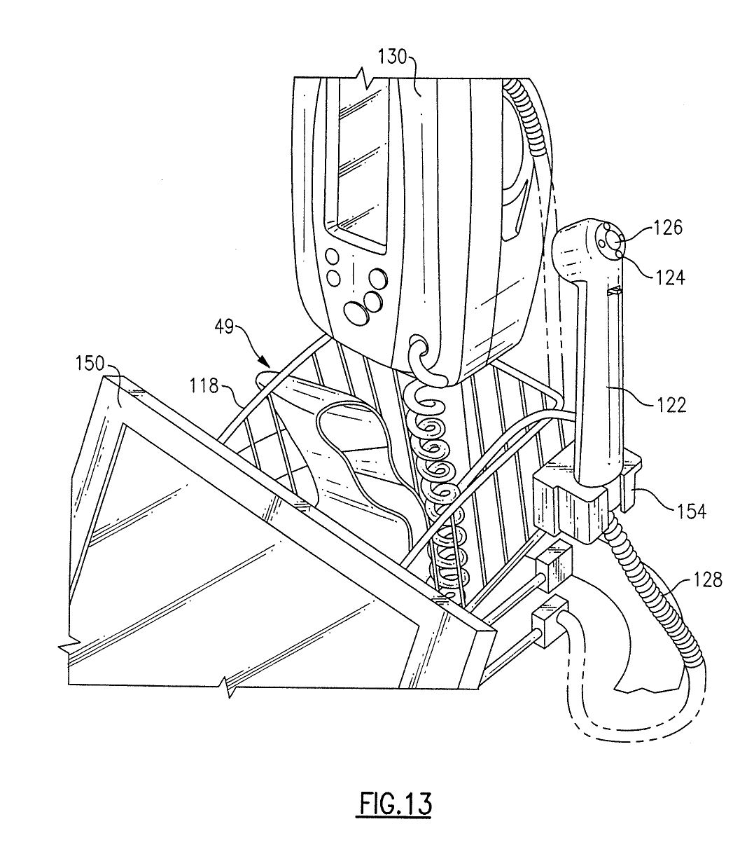

The vital signs collector 40 according to this embodiment is a SPOT™ vital signs monitor manufactured by Welch Allyn, Inc., of Skaneateles, N.Y., the monitor including a housing 41 having an integral display 42 to which a plurality of various probes can be attached, such as, an oral thermometer probe, a pulse oximeter probe, and a blood pressure cuff 49, as shown in FIG. 13, for evaluating certain physiological parameters (vitals) of a patient (not shown). The vital signs collector 40 further includes a rechargeable battery (not shown), as well as a wall transformer.

According to this specific embodiment, the supporting structure 20 of the herein described medical diagnostic workstation 10 is movable to permit location of same between a myriad of patient areas, the supporting structure including a lower portion 24 defining a wheeled base unit 26. The lower portion 24 and the upper portion 22 of the supporting structure 20 are separated by a vertically extending support post 28 to which other electronic equipment is attached, such as transformers, power supplies, and/or power strips. This support post 28 can preferably be axially adjusted so as to be extended or retracted to permit height adjustment of the diagnostic workstation 10.

More particularly, the upper portion 22 of the supporting structure 20 includes a horizontal platform 36 having a top supporting surface permitting the computing device 30 to be seated or attached thereupon, preferably in a releasable fashion. The housing 41 of the vital signs collector 40 is mounted in the vicinity of the computing device 30, also preferably in a releasable manner, on an adjacent planar support 48 and is separated therefrom by means of a wireless link 54 that permits bi-directional wireless communication between these components. The releasable attachment of the vital signs collector 40 to the planar support 48 affords versatility in that the vital signs collector 40 can easily be moved on its own as needed and used with its own internal power supply (not shown). The wireless link 54 according to this embodiment is an infrared link to a PCMCIA card, though it should be readily apparent that other known forms (optical, IRdA, RF, etc) of wireless interconnection can be utilized. The PCMCIA card (not shown) permits the vital sign data to be communicated from the computing device 30 to a local area network (LAN). Cables extending from the transformers of the computing device 30 and the vital signs collector 40, respectively, are connected to a corresponding isolation power transformer 64, that is supported by conventional means onto the vertically extending support post 28. This transformer 64 permits each of the computing device 30 and the vital signs collector 40 to be charged for a predetermined time interval.

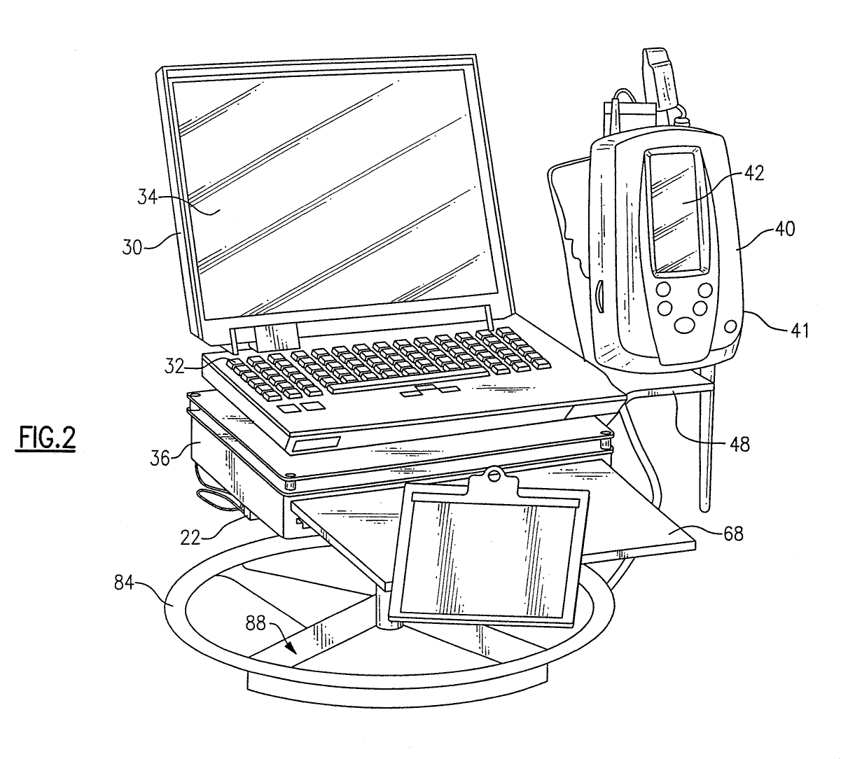

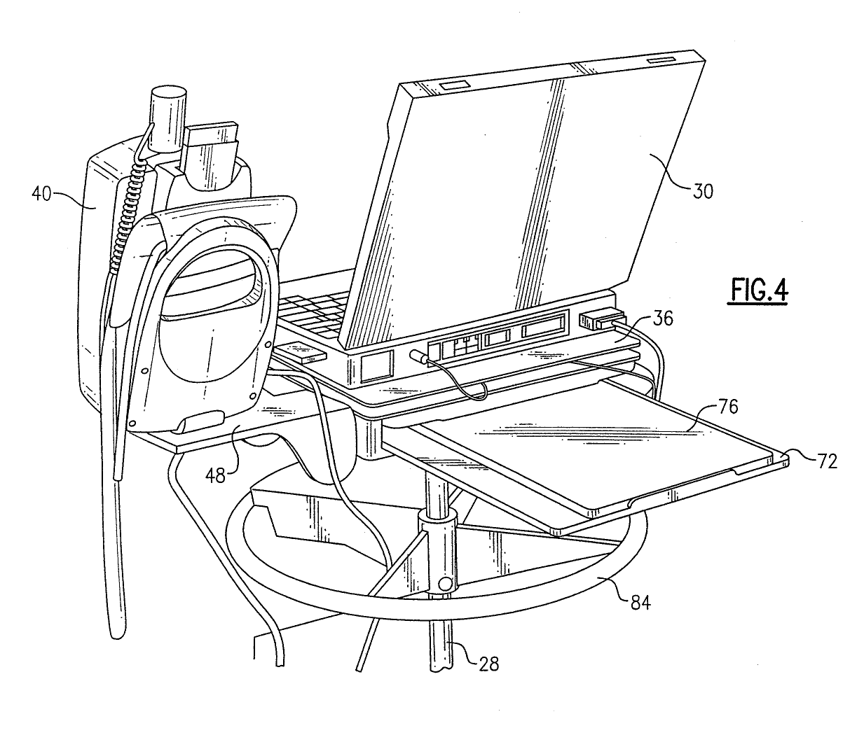

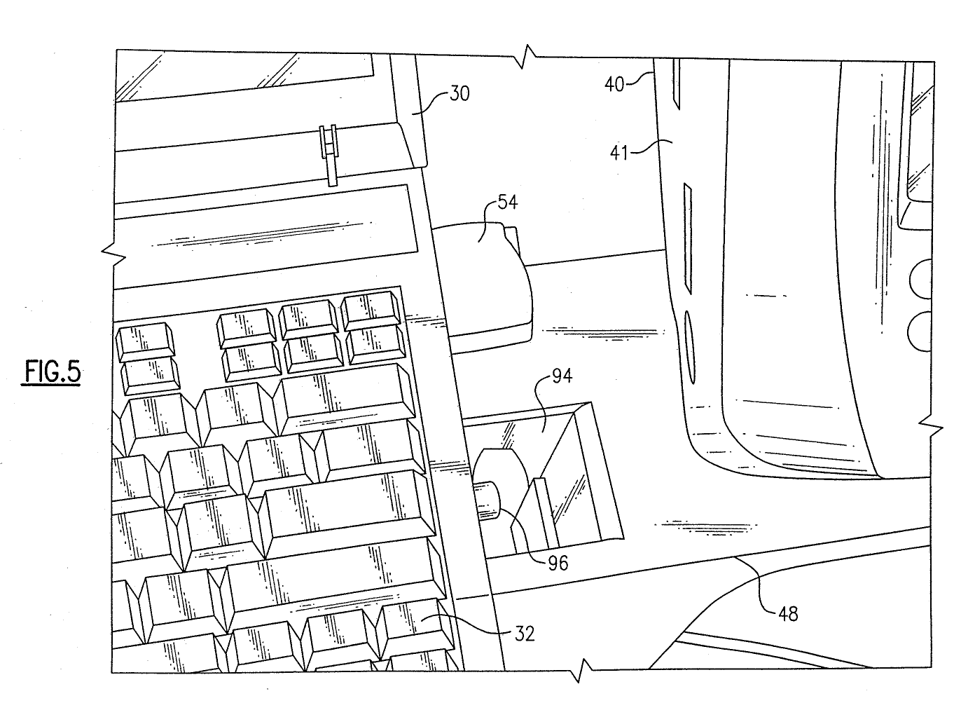

The horizontal platform 36 is defined by a supporting body having a pair of spring loaded horizontally extending sections 68, 72 that are located on respective front and rear sides thereof. The section 68 on the front side of the platform 36 extends to form a working surface for the user as shown more clearly in FIG. 2, while the section 72 on the rear side contains a spare battery 76 for the computing device 40, as shown more clearly in FIG. 4. Each of the sections 68, 72 retract normally into the interior of the platform 36 until needed. A supporting receptacle, in this instance, a basket 80, attached by conventional means to the support post 28, is used in conjunction with the vital signs collector 40 to retain a number of patient-related items, such as but not limited to the blood pressure cuff 49, FIG. 13, spare EKG or temperature probes, as well as disposable items such as probe covers, and the like.

The supporting structure 20 according to this embodiment further includes a gripping handle 84 projecting circumferentially about the support post 28 at an intermediate height between the upper and lower portions 22, 24. The gripping handle 84, that permits the supporting structure 20 to be easily transported between various patient areas, preferably includes at least one additional storage pocket 88.

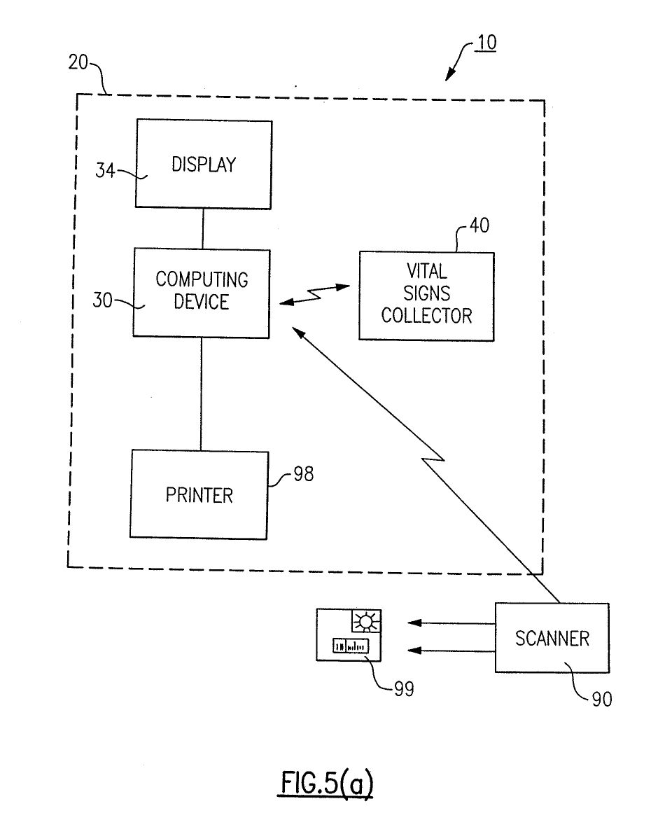

The lateral planar support 48 further includes an opening 94 for an IRdA or other wireless link 96 to permit the computing device 30 to communicate with a bar-code scanner 90 (shown only schematically in FIG. 5(a)). Alternately, and though not shown, a hard-wired connection could also be used. According to this specific embodiment, the scanner is a Dolphin 7400 Portable Data Terminal (PDT), manufactured by Hand Held Products, Inc. The above product includes scanning software which permits both one dimensional (1D) and two dimensional (2D) bar code symbologies (such as PDF 417, Maxi Code, QR Code and Data Matrix, among others) to be read and interpreted. In addition, this scanner 90 further includes an imager, such as a CCD as well as processing circuitry which also permits .GIF and .JPEG images of signatures, images, and ID cards, etc also to be taken.

In operation and referring to FIGS. 1-5(a), the bar code scanner 90 acting as the input device for the workstation 10 can be used initially to obtain information relating to the patient if there is no previous data stored in the memory of the computing device 30. For example, and in an emergency or triage setting, the scanner 90 can first be used remotely from the workstation 10 to scan a patient's ID 99 (such as a driver's license) to obtain demographic information and to scan an image. This information can be gathered in the doctor's office or examination room or obtained remotely and stored by the scanner 90. The information contained in the scanner 90 can then be transmitted to the computing device 30 through the wireless link 96. Similarly, a doctor or other user's badge can also be “swiped” using the scanner 90 and access can be granted or denied based upon a stored list of authorized users that are contained in memory. Otherwise, the diagnostic workstation 10 is powered by activating the computing device 30 and the vital signs collector 40.

Once the user has been identified and compared to a known list of authorized users, the doctor or other user is permitted to view the current patient data, if any, and to use the workstation 10. In a typical clinical setting, and if the patient is a “new” patient having no previous history, data can be entered, either through use of a bar code tag or other scannable identification means or through manual entry using the keyboard 32 or through the keypad of the scanner 90 with subsequent IR transmission to the computing device 30.

Upon the taking of patient demographic data as noted above, vital signs can then be taken (e.g., blood pressure, pulse rate, body temperature, SPO₂, etc.) using the vital signs collector 40 in the usual manner using each of the probe assemblies. The readings are displayed on the collector display 42 and are transmitted into the memory of the computing device 30 through the wireless link 54. The command to transmit the vital sign readings can be done automatically or through a command that can be manually entered into the keyboard 32 or by way of a specific control button (not shown). These results can then be displayed on the display 34 of the computing device 30, along with other information, including patient demographics and image data.

Optionally, a printer 98 (shown only in FIG. 5(a)) can be interconnected to the computing device 30 allowing any of the displayed information to be printed as needed, in the form of a summary sheet. This summary sheet is scannable; that is, including at least one machine-readable portion, e.g., at least one bar code, permitting the sheet to be carried to other examination areas along with the encoded information. The encoded information can include, for example, patient demographic data and/or physiological parameter data. In this manner, the summary sheet can be uploaded at a subsequent location, such as a billing station, a second physician's office, or an additional testing facility. At any of the latter, the bar coded information can be scanned and uploaded to a computer at that particular site. New information or changed information, such as a change of address, change of billing status, new medication data, etc., can be added to at least one code on the summary sheet and then the summary sheet can be printed.

In addition to the above, previous vital sign and or any other stored data relating to the patient (if an existing record exists) can be displayed and charted so as to present trending information to the care giver and downloaded (e.g., printed) or transmitted to a remote location, for example, using an Internet connection. According to the present embodiment, for example, the information can also be sent via the PCMCIA card over a local area network (LAN not shown) to a remote site, such as, for example, a central receiving station at a remote hospital.

In addition to previous vital sign and patient-related data, the bar code scanner 90 can be used to scan other information such as supply and inventory used in the care and treatment of a patient, including disposables and medications by scanning bar-coded information contained on each item used and storing the results into a resident database. This database can be accessed remotely, such as through a LAN interface, to indicate when supplies on the supporting structure 20 are depleted and need replenishing and for tracking items and procedures vis a vis the patient. The above can be charted and catalogued using the above workstation 10. Similarly, other information which can be obtained through scanning of machine-coded symbols and the like can also be tracked in a similar manner.

### SECOND EMBODIMENT

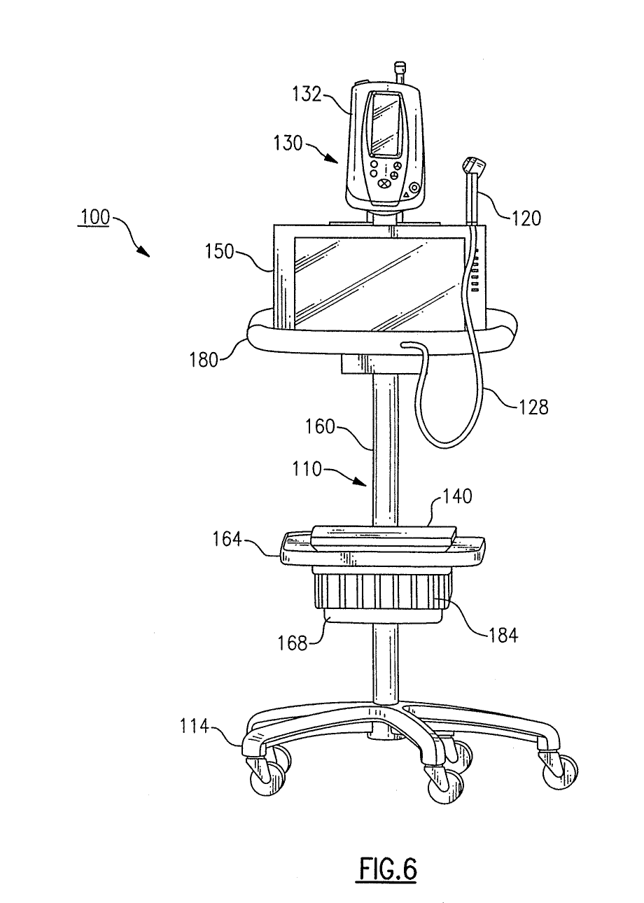

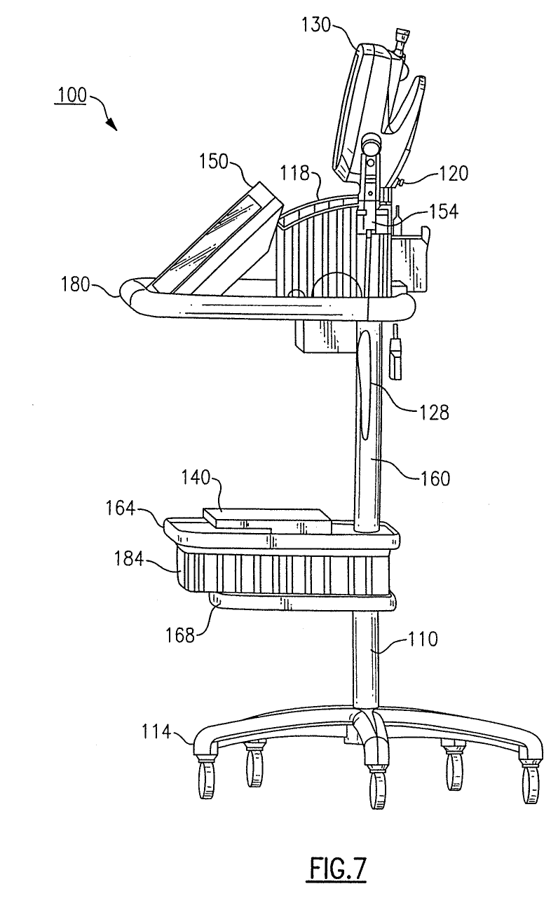

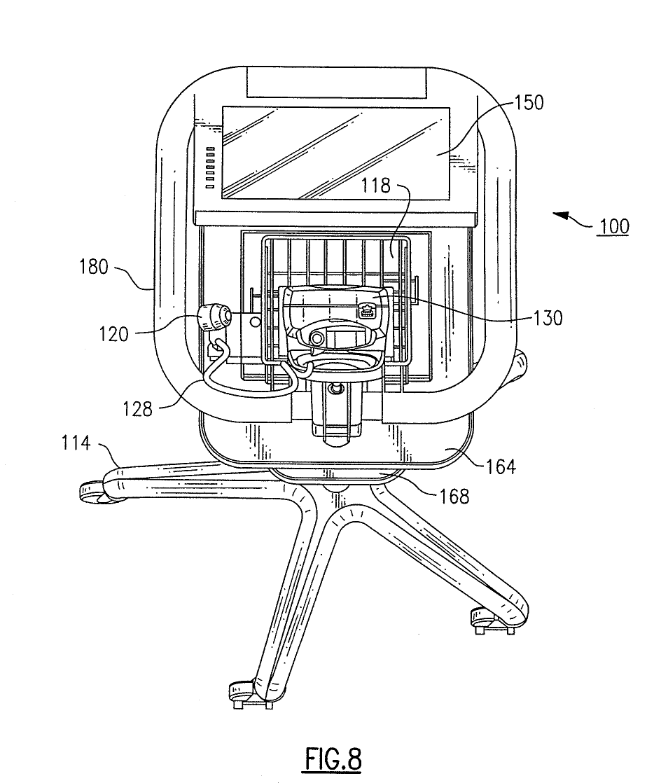

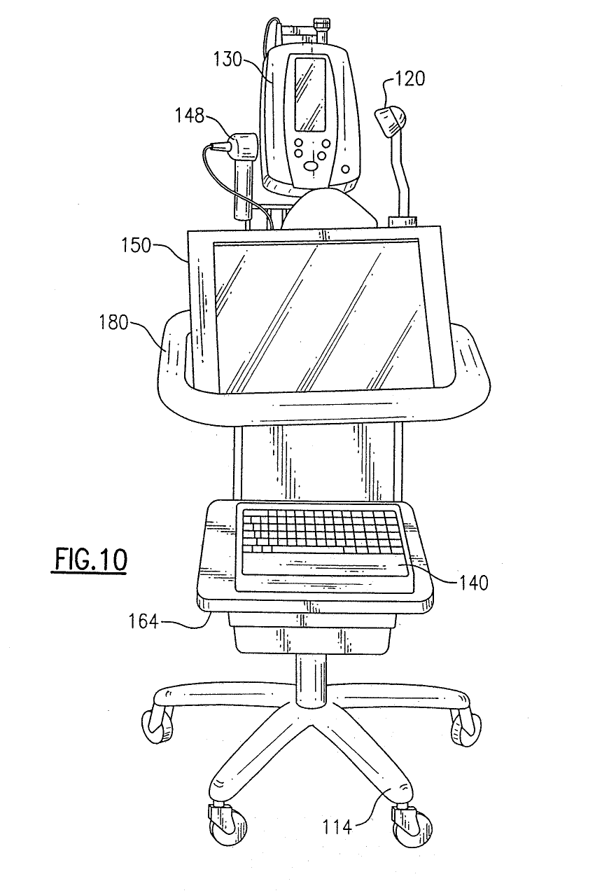

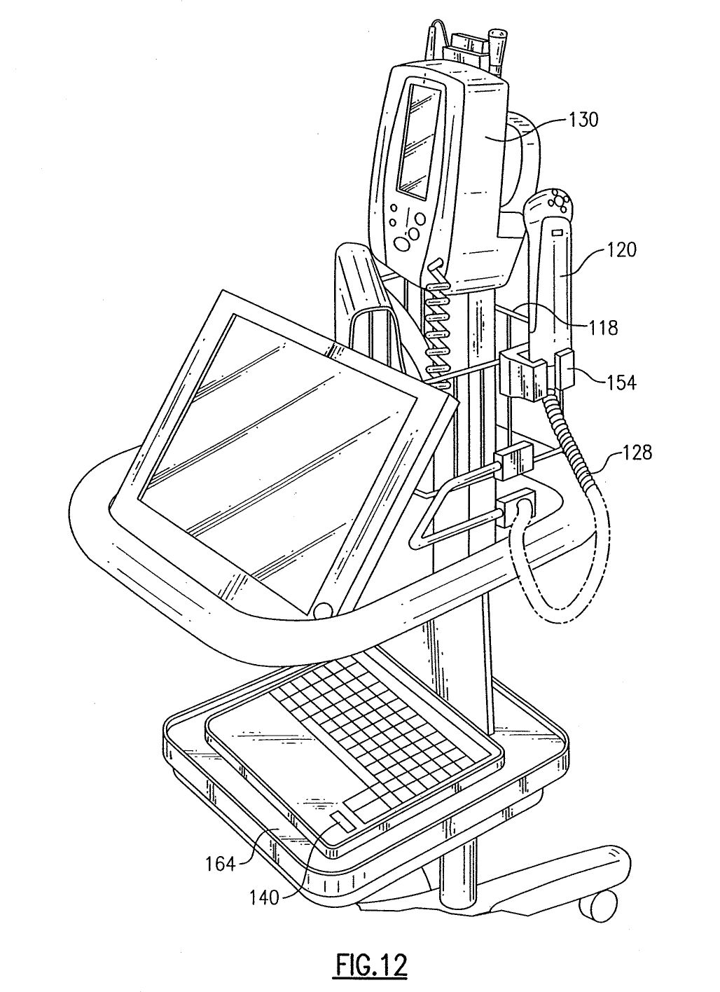

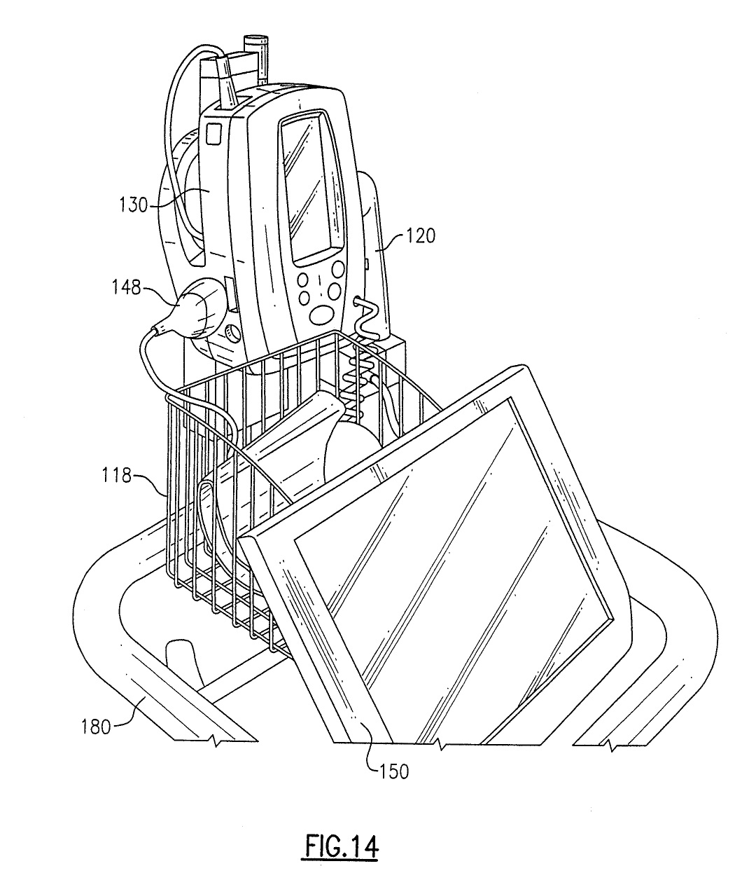

Referring to FIGS. 6-15, there is disclosed a medical diagnostic instrument workstation or platform herein designated by reference numeral 100 according to a second embodiment of the invention. As in each of the embodiments that are described herein, the workstation of the present invention includes a plurality of individual discrete components that are interconnected and retained, except where indicated, on a common supporting structure, thereby defining an integrated assembly. In brief and according to this specific embodiment, these supported components include a miniature imaging device such as a video camera 120, a vital signs collector 130, a portable computing device 140, and a display 150, each component being supported by a movable cart 110. The cart 110 includes a wheeled base 114 which permits movement between various areas, such as among patient rooms on a hospital ward, as well as a supporting receptacle such as a basket 118 that is arranged on a vertically extending support post 160. The support post 160 retains or includes means for retaining the discrete components of the workstation while the supporting basket 118 permits storage of a blood pressure cuff 49, FIG. 13, disposable items such as used probes and the like, or other patient related items. Each of the supported components will now be described in greater detail.

The miniature video camera 120 comprises a housing or handle 122 permitting handheld operation, including an electronic imaging element, such as a CCD or CMOS-type electronic imaging element, that is arranged within the interior of the housing relative to an objective lens system 126. Processing circuitry, including an A/D converter, permits an optical signal detected by a pixel array of the electronic imaging element to be converted into an electrical signal and the electrical analog signal to be subsequently converted into a suitable digital video signal. The specific details of digital signal video processing are very well known in the field and do not constitute an essential part of the invention, except through integration thereof.

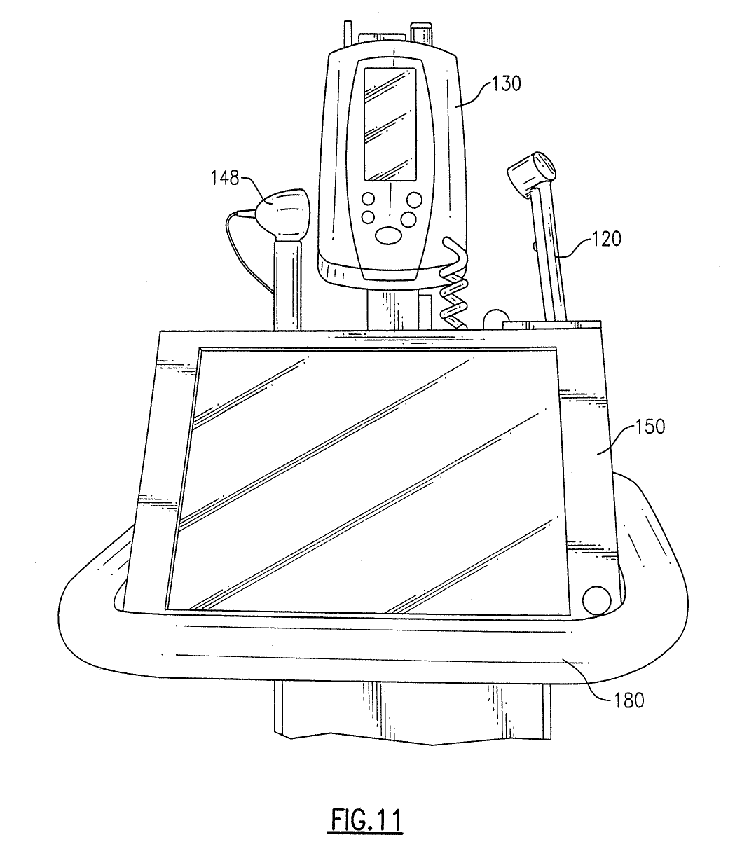

A tethered cord or cable 128 permits the miniature video camera 120 to be removed from a receiving cradle 154 provided on the workstation 100 for use. The miniature video camera 120 also preferably includes a plurality of white LEDs 124 to provide necessary illumination of a medical target, the LEDs being retained within an instrument head and arranged circumferentially in relation to the objective lens system 126. Alternatively, however, other illumination sources, such as, for example, miniature incandescent lamps, such as halogen lamps can be substituted. According to the specific embodiment, a magnetic switch (not shown) contained within the interior of the miniature video camera 120 engages a magnet (also not shown) disposed within the receiving cradle 154. The use of the magnetic switch permits the camera 120, including the white LEDs 124 and the electronic imaging element to be activated automatically when removed from the receiving cradle 154. The camera 120 can be left in a powered “on” condition by removing the camera housing 122 from the receiving cradle 154 and then reinserting same in a reversed orientation as shown in FIGS. 11-13. The camera 120 is electrically connected to the computing device 140 which includes sufficient nonvolatile memory for storing a plurality of processed digital images.

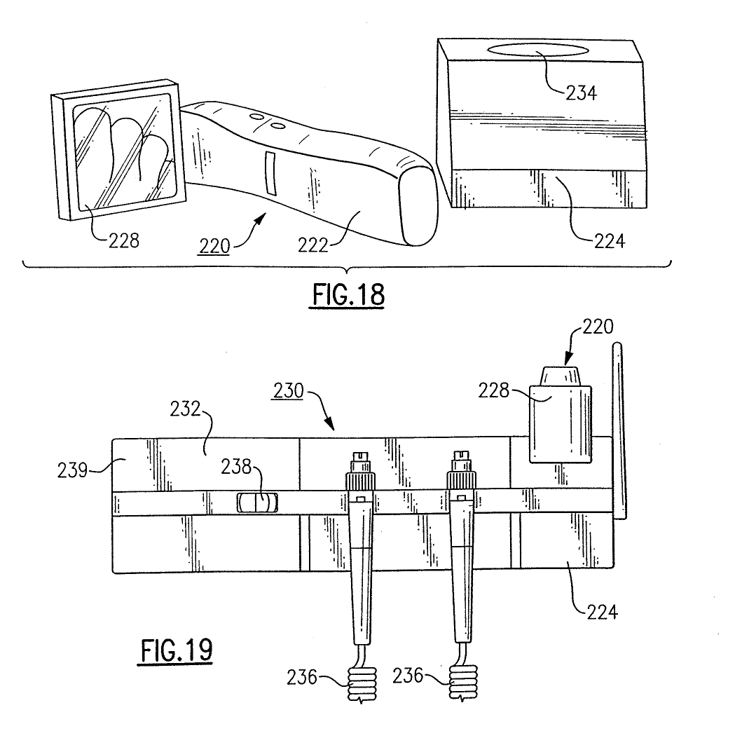

Though not shown in this embodiment, the miniature video camera can also include an integral display to permit the physician or other user added flexibility in the use of the presently described workstation. An example of a miniature video camera that includes an integral fold-out display is described in a later embodiment as shown in FIG. 18.

The vital signs collector 130 is attached relative to the top of the cart 110, preferably as a releasably attached item. As in the preceding, the collector 130 is a SPOT™ vital signs monitor manufactured by Welch Allyn, Inc. of Skaneateles Falls, N.Y. which is capable of measuring a number of patient vital signs including saturation blood volume, blood pressure, oral body temperature, and pulse rate using dedicated probes attached to a housing 132 having an integral display 134. It will be readily apparent to one of sufficient skill in the field, however, that literally any monitoring device capable of obtaining patient vital signs can be employed. Making the collector 130 releasable, as in this embodiment, improves the overall flexibility and versatility of the workstation 100 in that the collector used herein contains a housed rechargeable battery. Alternately, however, the collector 130 can also be fixed installed or manufactured integrally with the cart 110. The location of the vital signs collector 130 itself on the cart 110 is not essential for reasons which will become apparent from the following discussion.

According to this embodiment, the portable computing device 140 is supported in a lower portion of the cart 110 on a horizontal platform 164 that is attached through an opening to the vertically extending support post 160. As in the preceding, the computing device 140 used herein is a conventional laptop computer having a keyboard and LED display, but as will be apparent neither of the latter features are essential to the workings of the present embodiment.

The workstation display 150 is attached within the framework of a gripping handle 180 located in an upper portion of the cart 110 adjacent to the supporting basket 118. The gripping handle 180 extends outwardly from the support post 160 slightly beyond that of the horizontal platform 164.

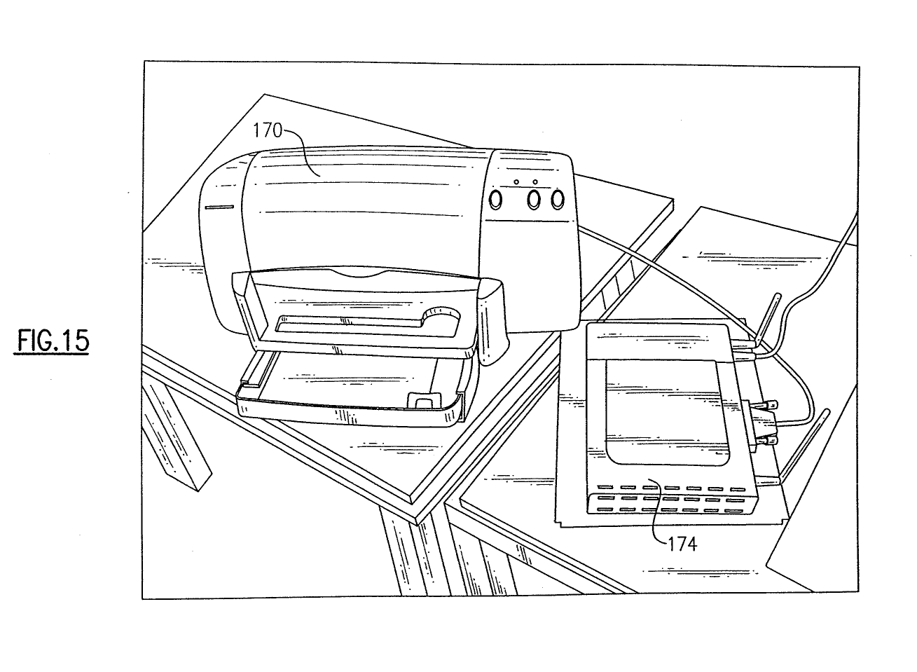

A printer 170 is positioned remotely away from the cart 110, for example in another portion of an office or patient room, and interconnected through a wireless link, such as a radio frequency (RF) connection through an access cell 174, with the portable computing device 140, as shown in FIG. 15. Other wireless protocols can similarly be utilized. The specifics of RF communication are known to those in the field and these specifics in and of themselves do not form an essential part of the present invention.

In terms of connectivity, the computing device 140 is electrically interconnected to the vital signs collector 130 and the workstation display 150, as well as to the miniature video camera 120. The workstation display 150 is used in lieu of the display (not shown) of the computing device 140 that is used in this embodiment. As noted above, the processor portion of the computing device 140 is all that is utilized as will now be described in terms of operation of this workstation 100. With regard to that processing ability and referring to this embodiment, preferably, each of the above components are electrically connected to the miniature video camera 120 to enable operation through use of a set of control buttons provided thereupon.

In this embodiment, the vital signs collector 130, miniature video camera 120, computing device 140 and workstation display 150 are electrically powered by a dedicated power supply (not shown) supported on the cart 110 within an enclosure 184 and supported by a parallel horizontal platform 168 located immediately beneath the platform 164. A wireless link 148 is established between the vital signs collector 130 and the computing device 140. In the present instance, the link is an infrared (IRdA) connection, though other known forms can be utilized.

According to this particular embodiment, the portable computing device 140 contains software which permits one dimensional or two dimensional bar code symbologies such as PDF 317, MaxiCode, DataMatrix, etc. or other machine readable information such as OCR (optical character recognition) to be identified and decoded from a captured digital image. The software that is used according to this embodiment is described more completely in U.S. Pat. No. 6,015,088, the entire contents of which are herein incorporated by reference. The computing device 140 is further programmed with software which enables operation of at least portions of the workstation 100.

According to the present embodiment, the housing 122 of the miniature video camera 120 includes a pair of exterior control buttons 142, 146 which enable substantial operation of the workstation 100. In brief a first control button 142, when depressed, enables a plurality of menu-driven functions as viewed on the workstation display 150, including image capture. This control is effectuated through an interface whereby the miniature video camera 120 is physically interconnected to the portable computing device 140 as a computer peripheral. The enabling interface circuitry for this connection is also contained along with the transformers, power cables, etc., within the enclosure 184 that is located beneath the computing device on platform 168, along with the onboard power supply (not shown) for powering each of the components retained by the workstation 100.

The above interface facilitates operation of the workstation 100 in that, according to this embodiment, an initial depression of the first control button 142 enables access to a set of menu items that are displayed on the workstation display 150 with a first click and subsequent execution of a particular menu item is accomplished with a second click, in the very same manner as a conventional computer mouse. A second adjacent control button 146 provided on the handle 122 of the camera 120 is used to toggle back through the displayed menu, such as to undo previous commands, including deletion of images as needed.

In operation, the miniature video camera 120 is first accessed by the user (not shown) and the first control button 142 provided on the camera handle 122 is depressed to initiate power-up of the workstation 100. The miniature video camera 120, which is automatically activated once released from the receiving cradle 154 by means of the magnetic switch being shifted based on movement away from the magnet contained in the receiving cradle, is then pointed at a doctor's badge (not shown) which contains machine-readable information and the image of the badge is captured by double-clicking the control button 142. The machine-code identification software contained within the computing device 140 then identifies the presence of machine-readable information (bar code, optical characters, etc.) in the captured image and decodes same. This decoding permits subsequent use of the workstation 100 by the user. Alternately, a finger-print or other biometric verification system can be used to identify the user and patient, based upon stored records, and thereby providing proper authorization and access for the workstation 100. An example of same is shown in a subsequent embodiment; see, for example, FIG. 25.

The patient can then be examined using the above-described workstation 100 wherein the patient's history/records (if existing) can be accessed after identification of same and proper authorization of the user. Vital signs data (blood pressure, pulse rate, body temperature, etc.) can be obtained using the probe assemblies of the vital sign collector 130 and the results can be transmitted to the memory of the computing device 140 through the interconnected wireless link 148 therebetween. These readings can be transmitted automatically or through actuation of the control button 142.

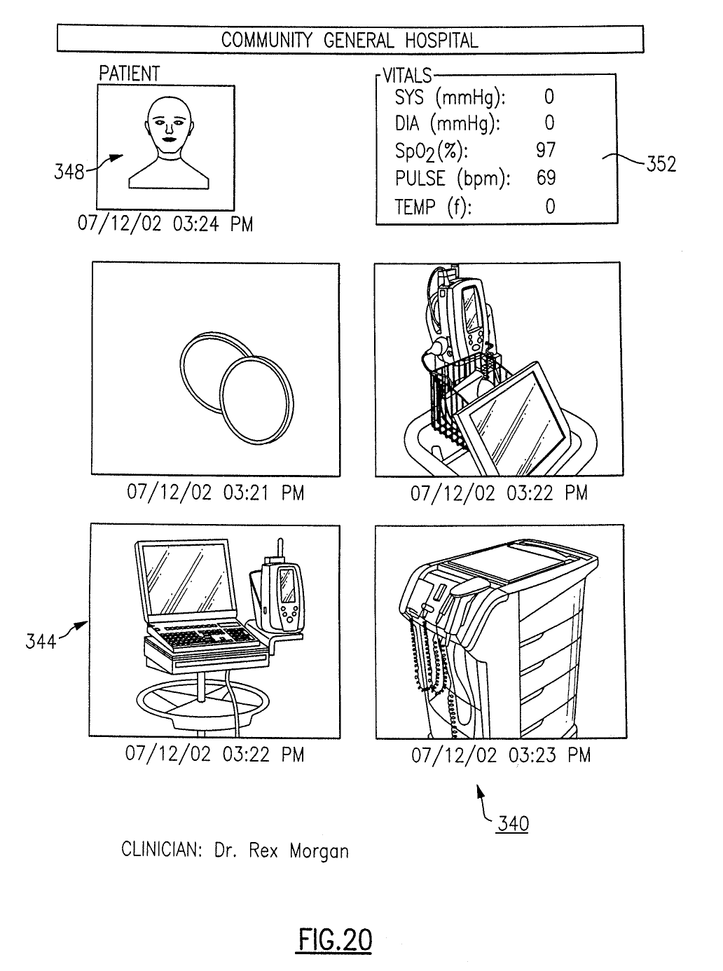

A blood pressure cuff 49 can be stored in the supporting basket 118 provided on the support 110 along with other instruments and other patient-related articles, such as disposable probes, which are not integrated with the workstation 100. A paper summary data printout, such as shown in FIG. 20, of displayed information, including a number of image prints and vital sign data, can be created and output via the printer 170 through an appropriate command using the control button 142 provided on the camera housing 122 based upon a menu selection which can be selected on the workstation display 150.

It should be apparent that many variations are possible. For example, the display of the computing device 140 could have been used in lieu of the separate display 150. Moreover, the vital signs collector 130 or another device could have been configured to control the operation of the workstation 100 rather than the miniature video camera 120. The miniature video camera 120 could also have been connected to the workstation 100 via a wireless connection rather than using tether cord 128. Other similar variations may be evident.

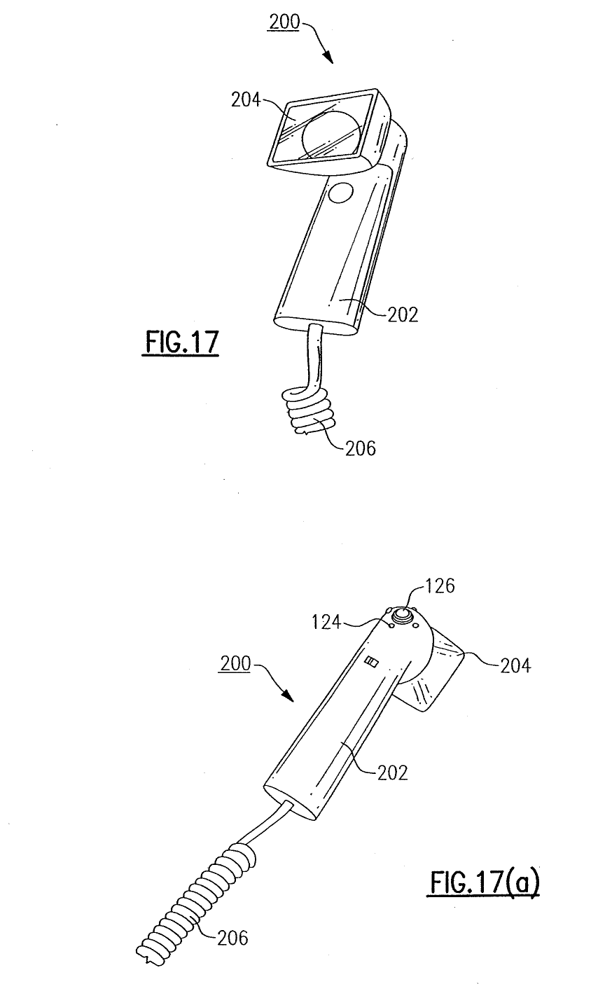

Referring to FIGS. 17 and 18, a pair of miniature video cameras in accordance with varied design in accordance with the invention are herein described. A first camera 200, FIG. 17, is similarly connectable to a receiving cradle for a workstation (not shown) such as previously described above. As in the preceding, the camera 200 includes an electronic imaging element (not shown) which is disposed in an instrument head of a handle 202 along with an illumination system and processing circuitry for producing a video signal. Rather than using a series of white LEDs as in the preceding, an integral display device 204 can produce a continuous stream of images to the user in lieu of an LED or lamp-type illumination system by using organic electroluminescent devices (OLEDs), such as those described in U.S. Pat. Nos. 5,684,365, 5,937,272, and 6,424,093, the entire contents of which are herein incorporated by reference. The instant camera 200 is tethered by a cable 206 to the remainder of the workstation (not shown) including a computing device having means for receiving image data from the camera. According to this embodiment, displayed images can be captured and transmitted to the computing device as previously described through a wired or wireless interface.

The display can either be integral to the body or head of the camera according to FIG. 17, or alternately can be deployed so as to “fold-out” or deploy relative to the body of the camera as shown in the embodiment of FIG. 18. In the latter design, a miniature video camera 220 includes a hand-holdable body 222 that includes a contained power supply, such as at least one lithium-ion or nickel-cadmium battery, which can be recharged by nesting same into the receptacle 234 of a docking/recharging station 224. The body 222 includes a hinged display 228 which, according to this embodiment, also includes an electronic imager and an illumination system which includes organic electroluminescent devices, such as those previously incorporated above. The docking/recharging station 224 can also include means for downloading at least one stored image which has been captured into memory by the miniature video camera 220.

In a further refinement and as shown in FIG. 19, the camera 220 and docking/recharging station 224 can be added as a releasable or fixed portion of a wall-mounted medical diagnostic workstation 230 having a transformer 239. In addition to retaining the miniature video camera 220 and docking station 224, the workstation includes a frame 232 having means for retaining a number of hand-held diagnostic medical instruments 236, such as ophthalmoscopes and otoscopes. The transformer 239 includes a switch 238 which, when activated, provides electrical power to each retained component.

### THIRD EMBODIMENT

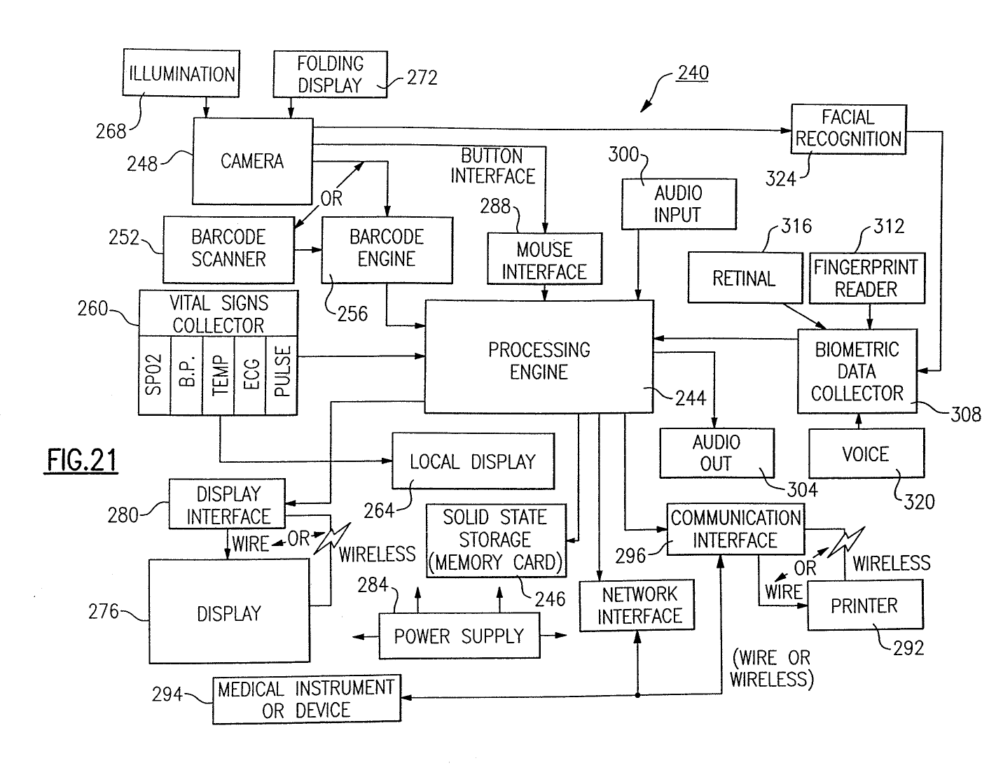

A functional block diagram of a medical diagnostic workstation 240 according to a third embodiment of the invention is now herein described according to FIG. 21. The workstation 240 is defined by a support (not shown) such as shown in any of the preceding embodiments which includes a number of components linked (that is, interconnected) through a processing engine 244. The processing engine 244, as previously stated, can be part of a portable laptop computer, or a Personal Data Assistant (PDA), such as a Palm® or other similar device. More particularly, the engine 244 is not confined to structure but to function simply through an I/O processor card having adequate solid state storage 246.

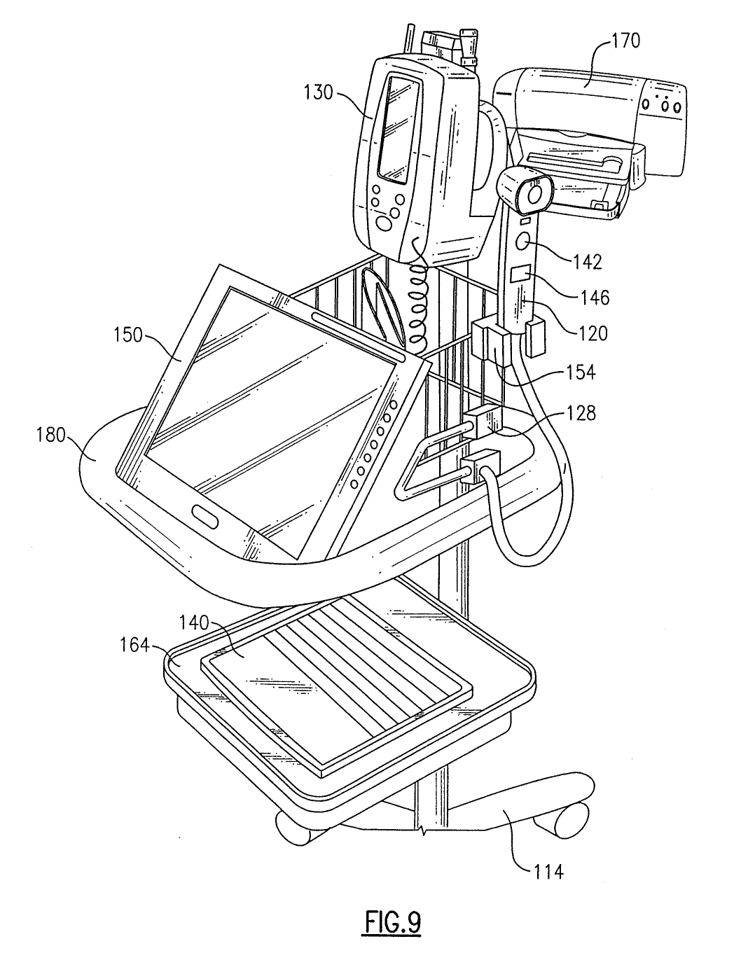

A number of discrete components are selectively incorporated with the above processing engine 244 including a camera 248 such as those described in FIGS. 9-18 and/or an input device, such as a bar code scanner 252 that includes a control interface/engine 256. The camera 248 preferably includes means for obtaining at least one electronic image of a target of interest and maneuver includes an illumination system 268 and optionally an integral display 272, such as the fold-out version as shown in FIG. 18.

Also linked to the processing engine 244 is a vital signs collector 260 capable of collecting vital signs readings of patient physiological parameters such as ECG, pulse rate, blood pressure, body temperature and SPO₂, among others, the collector preferably having a local display 264.

A display interface 280 and workstation display 276 are also connected to the processing engine 244, wherein the interface can be defined by either a hard wired or wireless link. Each of the above components are interconnected to a power supply, shown schematically as 284.

As in the preceding embodiment, the camera 248 can be attached to the processing engine 244 in the manner of a peripheral device through what is referred to as a mouse interface 288, due to the nature of the controls added to the camera in the form of buttons which operate in a double click manner to control operation of the workstation 240.

A printer 292 is also attached to the processing engine 244 through a separate interface 296 which can be hardwired or wireless (RF, IRdA, etc.) to permit image and/or vital sign reading and other data to be outputted as needed.

According to this embodiment, audio data can be added through a microphone or other input means 300 to the processing engine 244 which can similarly output through means 304 via a speaker (not shown) or provide other output storage such as corresponding wav.-files for subsequent retrieval. Alternately, audio data can be input and output using a cellular telephone connection through a network interface.

A biometric data collector 308 links to the processing engine 244 whereby specific authorization is guaranteed only through a particular biometric which can include finger prints 312, retina or iris scan 316, voice encryption 320, facial recognition 324 using the camera 248 and/or from bar-encoded information retrieved from the bar code scanner 252.

In operation and according to this embodiment, either a bar code scanner or camera (or other image capture device) can be used. Alternately, both forms of device could also be incorporated together depending on the type of control and information/application required. For purposes of this discussion, each portion will be discussed separately.

First and as to a workstation version utilizing the camera 248, the camera housing includes a plurality of button controls through its mouse interface 288 with the processing engine 244 that enables substantial operation of the workstation 240 by scanning a menu provided on the display and subsequent actuation of the appropriate control button.

The camera 248 can be connected to the bar code engine 256 such that any machine-readable information contained in an image captured by the camera can be detected and then automatically decoded. This feature can be used for encryption purposes, for example, to identify an authorized user (e.g. a health care provider) or to retrieve other information, such as, for example, patient demographics rather than relying upon manual entry of same.

The workstation 240 can also or alternately use the biometric data collector 308 to provide means for preventing unauthorized use through detection of a prescribed biometric, such as those, but not limited to those, listed on the block diagram. All of the componentry (printer 292, vital signs collector 260, display 276) are all preferably interconnected through their various interfaces to the processing engine 244 and ultimately through software contained therein to the camera button control. The display 276, once authorization is achieved, presents a menu that can be toggled through by selective actuation of the control of the camera 248, including collection of vital signs, capture of various image data, outputting of summary reports containing both image data and vital sign data, including if requested, trend data. A sample of a summary data sheet 340 is presented in FIG. 20 that includes a plurality of captured images 344 selectively displayed as well as a patient image and a patient information window 352.

The bar code scanner version is somewhat similar in that this device 252 through its interface 256 with the processing engine 244 controls the operation of the workstation 240. In addition, and as described in succeeding embodiments herein additional capabilities are brought to bear using a scanner controlled version.

In brief, the scanner 252 is linked to the processing engine 244 as are the remaining components previously noted. A set of instruction commands executable by the workstation 244 includes various commands including scan, print, capture image, obtain vital signs, obtain specific “vital sign”, etc. that can be contained either on a separate sheet or table in the vicinity of the scanner 252, which is tethered or wirelessly connected to the workstation 240.

Other medical instrument or device 294 can be connected to the processing engine 244 in order to receive types of medical data relating to a patient encounter, including but not limited to image data, text data, calibration data, and instruction sets including maintenance instructions.

As each instruction command is scanned, the processing engine 244 decodes the instruction and executes the command automatically through its separate interconnections with the remaining components.

### FOURTH EMBODIMENT

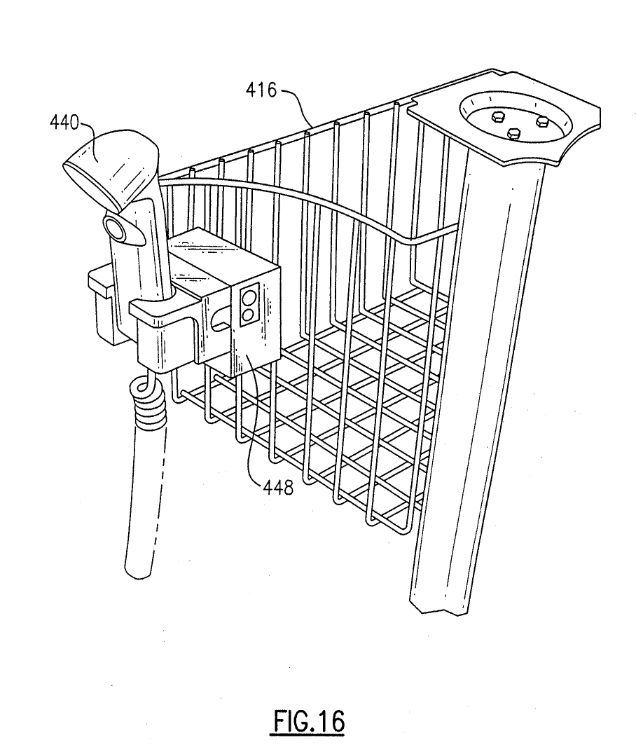

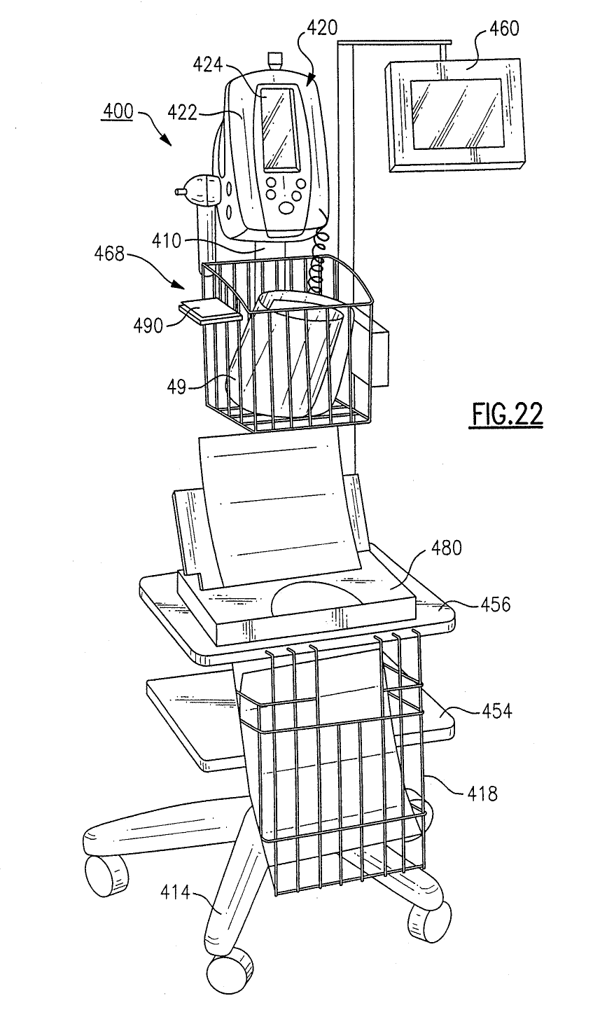

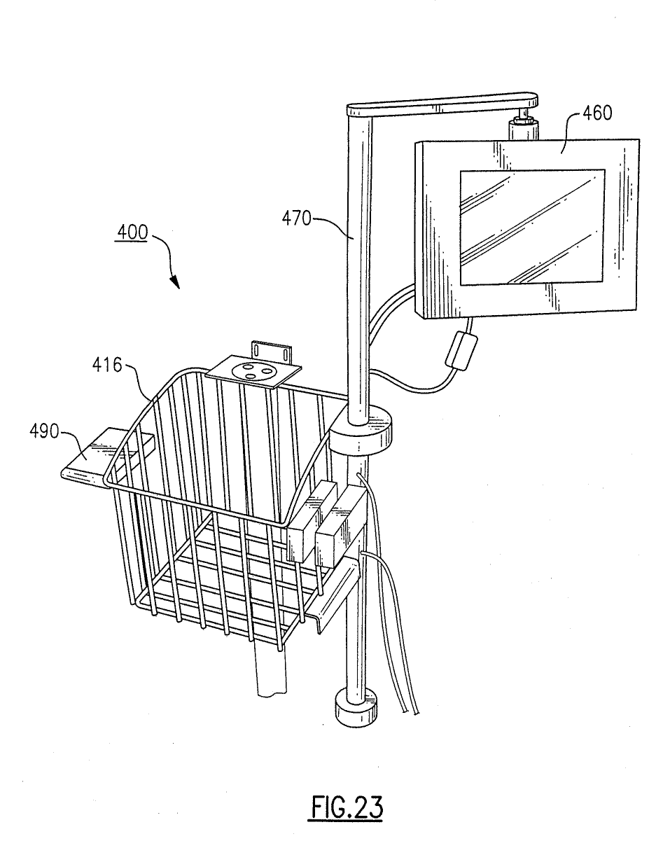

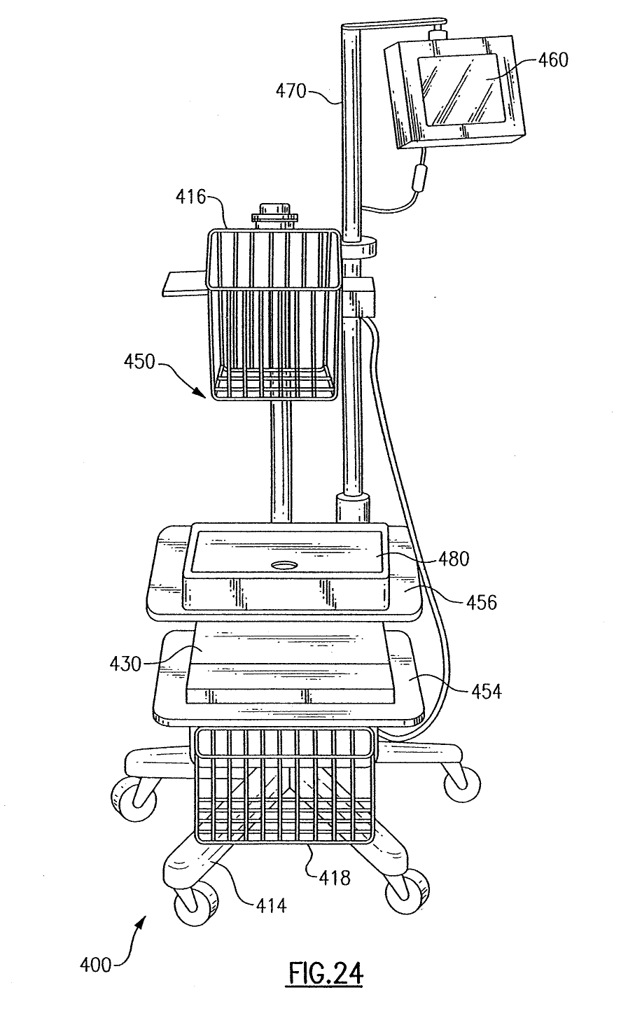

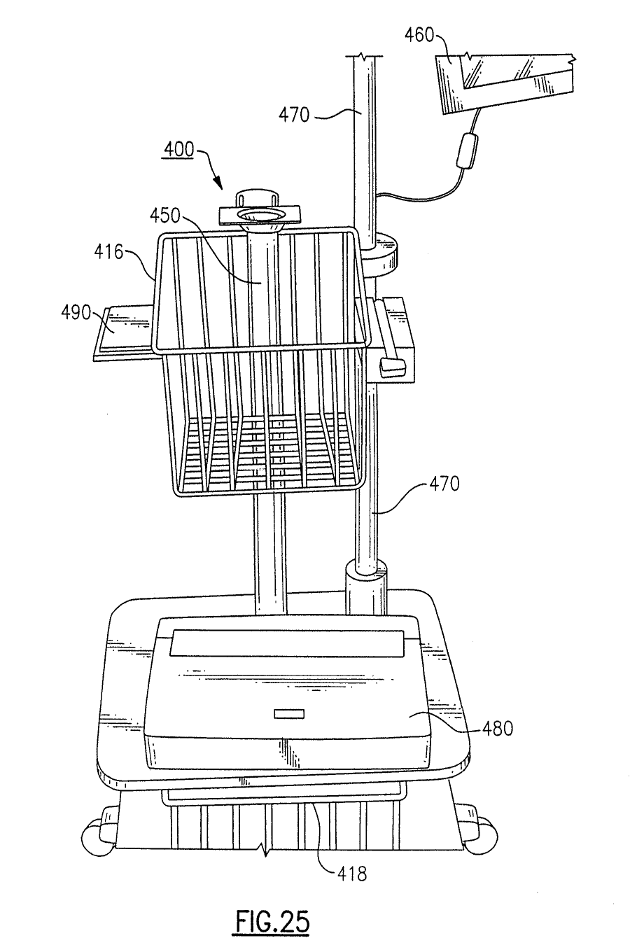

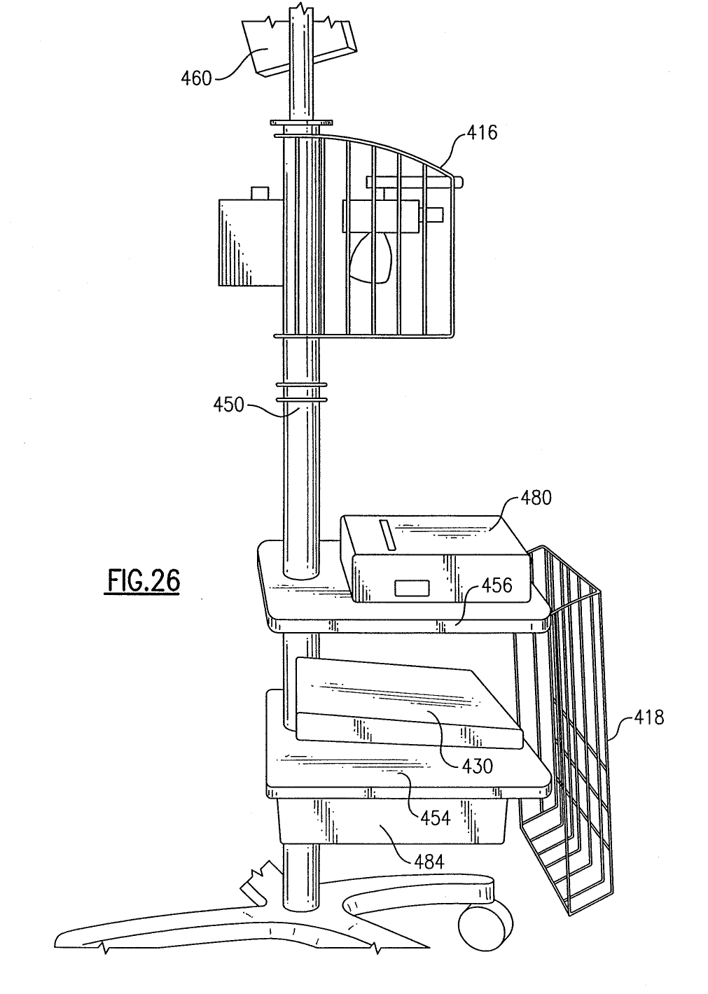

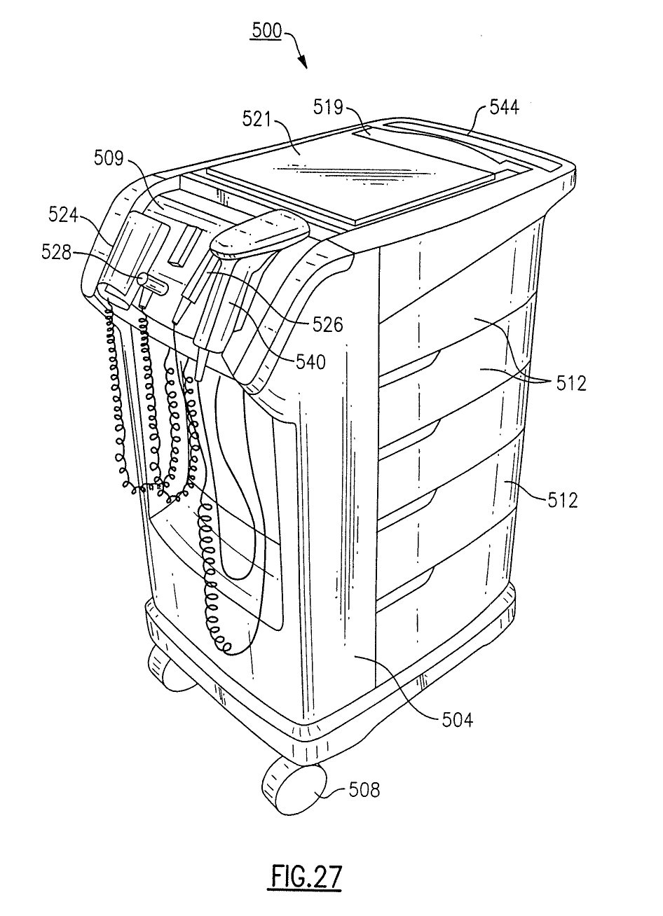

Referring to FIGS. 16 and 22-26, a medical diagnostic workstation 400 in accordance with a fourth embodiment of the invention is herein shown and described.

In brief, this medical diagnostic workstation 400 is similar to the imager-based workstation 200 previously described, but with a somewhat different configuration to illustrate versatility.

Like the preceding, the presently described workstation 400 is defined by a movable support 410 having a wheeled base 414 that maintains and supports a number of discrete components. These supported components include: a vital signs collector 420, a computing device 430, a miniature video camera 440, shown only in FIG. 16 as retained in a receiving cradle 448, a workstation display 460, and a printer 480.

Like the preceding, the vital signs collector 420 according to this embodiment is a SPOT™ vital signs monitor which includes a compact housing 422 and a local display 424, the monitor having probe means for measuring blood pressure, blood saturation volume, pulse rate and body temperature of a patient. The computing device 430 according to this embodiment is a portable laptop computer that includes a Pentium processor with adequate solid state memory as well as a keyboard and a display, though neither of these two latter features are actually utilized or required in this embodiment. That is to say, only the processing engine of the laptop computer is actually all that is required for use by the workstation 400.

Each of the vital signs collector 420, computing device 430, and miniature video camera 440 are attached to a vertically extending support post 450 of the movable cart 410, similar to that described in the preceding embodiment. Also and as in the preceding, the vital signs collector 420 is preferably releasably attached to the top of the support post 450 and the computing device 430 is retained on one of a pair of parallel horizontal platforms 454, 456 attached to the support post 450. Unlike the preceding, however, a second support post 470 that is arranged parallel to post 450 retains the workstation display 460, which is attached by conventional means to the top of the post. A pair of supporting baskets 416, 418 are provided, the former being provided adjacent the top of the support post 450 and the latter being arranged between a pair of horizontal platforms 454, 456 on the front side of the workstation 400.

Each of the supported components 420, 430, 440, 460 and 480 are interconnected structurally and electrically to define an integrated assembly. The vital signs collector 420 is connected to the computing device 430 through an IRdA or other suitable wireless link 468 to permit transmission of stored vital sign readings which can be displayed along with the image data on the workstation display 460. Though the display of the computing device 430 could be used to display this data, the workstation display 460 is used for this function. The printer 480 is provided on the platform 456 on the cart 410 through a wired connection or as previously shown a wireless connection could also be established, as in FIG. 15 between a printer and the remainder of the workstation 400, selectively, to output a hard copy of patient data, on command. Preferably, all or most controls of operation of the workstation are performed through an interface which similarly makes the camera 440 appear as a peripheral device to the computing device 430. As in the preceding, this interface circuitry as well as an onboard power supply and other transformers and electrical equipment are retained within an enclosure 484 disposed beneath the computing device 430 on the workstation 400.

In addition, the herein described diagnostic workstation 400 includes a fingerprint verification system 490, such as those manufactured by Sony Instruments, Inc., which is preferably attached in an upper portion of the support and is interconnected to the computing device 430. Preferably, a plurality of authorized fingerprints are stored on file and compared to those being evaluated in order to allow only authorized users to access the workstation 400.

In operation, the workstation 400 is used in a similar manner as the preceding. The miniature video camera 440 is removed from its receiving cradle, thereby activating the camera which is used to capture an image of the patient, if needed. In the meantime, the finger print verification system 490 permits authorized access to the workstation 400 through comparison of the user's fingerprint with that of a stored list of authorized users.

The control buttons of the miniature video camera 440 are used to operate the workstation 400. Vital sign readings can be taken in a conventional manner using the vital signs collector 420 with the results being transmitted wirelessly to the memory of the computing device 430 and then selectively displayed.

Images can be captured using the camera 440 and added to the stored patient record and also displayed in addition to the vital sign data. All of the results can then be selectively outputted to the printer 480 through operation of the control buttons as guiding through a menu appearing on the display.

### FIFTH EMBODIMENT

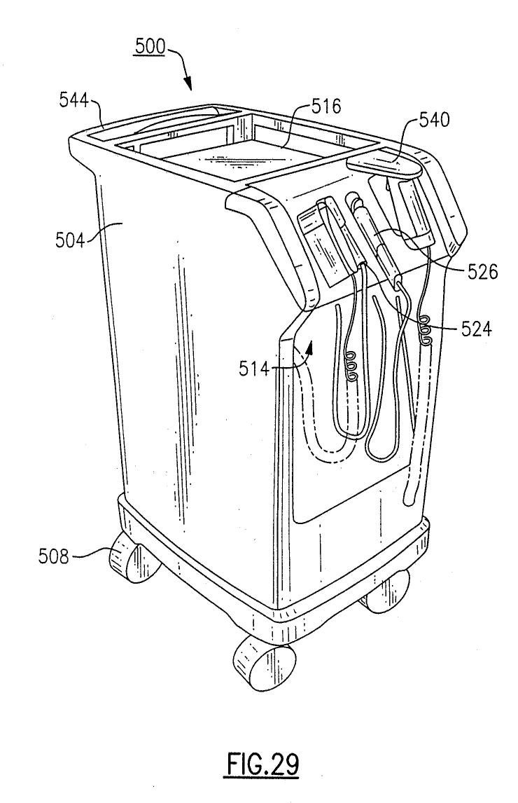

Referring to FIGS. 27-32, a medical diagnostic workstation in accordance with a fifth preferred embodiment of the present invention is herein described.

The medical diagnostic workstation 500 of this embodiment includes a movable cart 504 having a base that includes a set of wheels 508. As opposed to the preceding embodiments, however, this cart 504 is defined by a cabinet-like structure that includes a top support 516 as well as a plurality of drawers 512 built into each lateral side of the cart 504 to provide storage of various patient-related items used in a hospital or clinical setting, such as spare EKG, temperature, or other probes, spare equipment, peripherals, and medications, as well as disposable items such as gloves, probe covers and the like. It is contemplated, for example, that a single drawer 512 of the cart 504 could be allocated per patient on a hospital floor. Preferably, each of the drawers 512 could include a lock (not shown) to prevent theft.

The movable cart 504 further incorporates and retains a number of integrated discrete components including: a vital signs collector 520, shown only in FIG. 31, such as the afore mentioned SPOT™ vital signs monitor, previously discussed, which is disposed within the confines of the cart, and a computing device 530; in this instance, a portable laptop computer having a keyboard 532 and a display 534 that is retained on the top support 516, wherein each of the above are not only supported by the cart but which are also electrically interconnected together. Each of the individual probe assemblies for the vital sign collector 520 are retained along a front facing side 509 of the cart 504 in separate receptacles, including a blood pressure cuff 524, a pulse oximeter probe 526 and an oral temperature probe assembly 528. Though not shown, it should be readily apparent that other probe assemblies, such as EKG probes and the like, can easily be incorporated into this workstation 500.

Integral to the workings of this embodiment, a bar code scanner 540, such as the IT 4410 2D CCD barcode scanner manufactured by Hand Held Products, Inc., is also electrically connected to the computing device 530 in a manner that is described in greater detail below and retained in a holster-like receptable provided on the front facing side 509 of the cart 504 adjacent the top support 516.

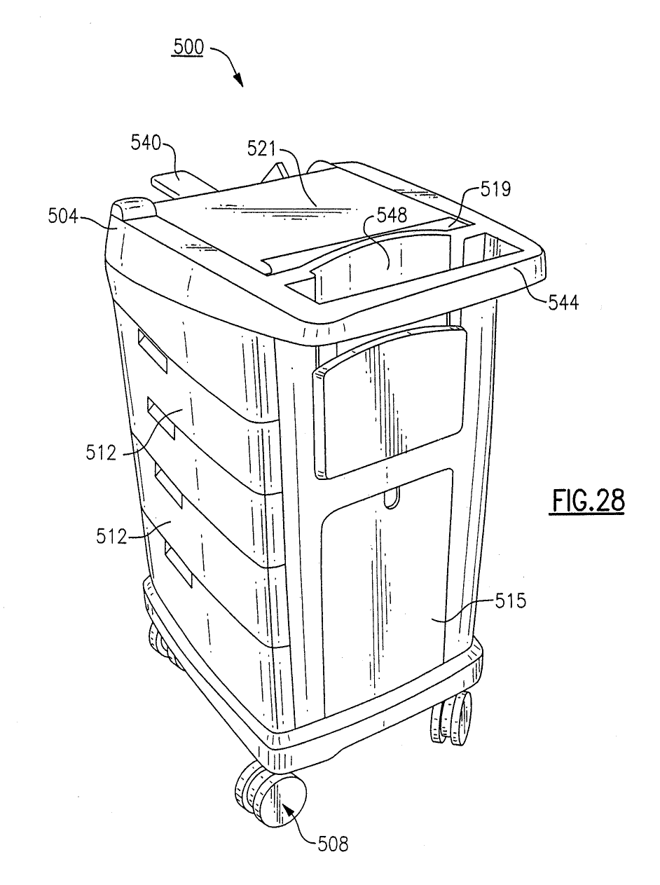

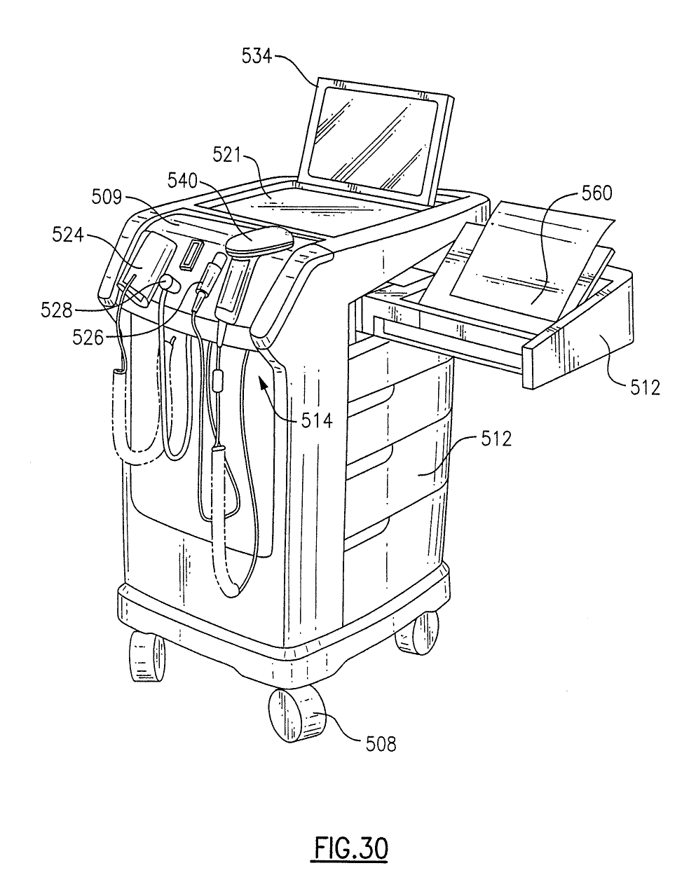

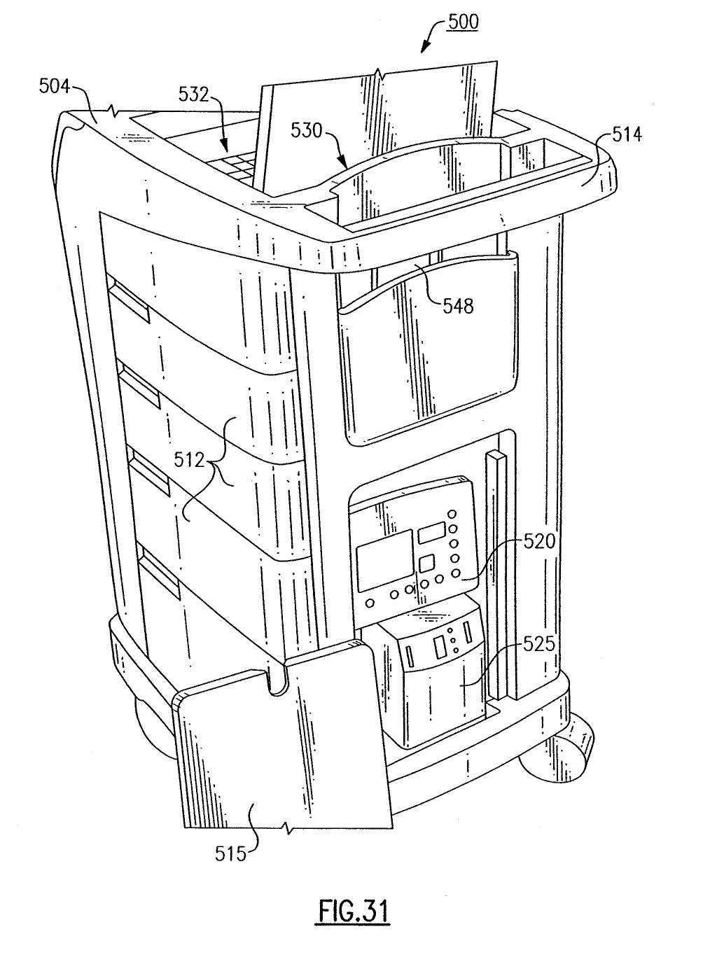

The computing device 530 is fitted into the top support 516 that includes a cavity which is appropriately sized for receiving the device and further includes a cover or working surface 521 that is hingably attached in order to cover the keyboard 532. An access slot 519, FIG. 28, permits the display 534 to extend therethrough, as shown in FIG. 30. As such, the computing device 530 is ostensibly hidden from the user, other than the display 534, and the user is almost unaware that the workstation includes the computing device, though the keyboard preferably remains accessible as a backup input/control device option.

The movable cart 504 further includes an internally contained power supply 525 contained therein, as shown only in FIG. 31, the supply being advantageously positioned beneath the vital signs collector 520, wherein each of the vital sign collector and power supply are accessible through a removable rear panel 515, shown as assembled in FIG. 28. Preferably, the onboard power supply 525 includes means for not only charging the internal power supplies (not shown) of the vital sign collector 520 and the computing device 530, but also for supplying power to the bar code scanner 540. The front facing side 509 of the cart 504 also includes a recessed portion 514 located beneath the storage receptacles for the probe assembles and the bar code scanner 540 to allow the extension cords for each to dangle without interference.

The herein described diagnostic workstation 500 maintains a total overall footprint which enables same to be moved easily between a plurality of patient examination rooms, for example, in a hospital or clinical setting, the cart 504 having a rear side gripping handle 544 that facilitates transport and a rear slot 548 adjacent the top support 516, that is sized preferably for storing a patient chart. Preferably, a set of machine-coded symbols 550, each preferably containing an encoded instruction command for operating the diagnostic workstation 500, is attached or otherwise made accessible to the scanner 540, as will be described below. This set of symbols 550 is acted upon solely by operation of the scanner 540 and the computing device 530 to enable operation of the workstation 500 without keyboard or other manual intervention by the user, significantly reducing the incidence of potential errors and permitting less sophisticated users to effectively service patients.

Referring to FIGS. 27-32, the above workstation 500 can operate as follows: First, a tag or identification ID of a patient (not shown) is initially scanned by the bar code scanner 540. As a result, certain information is accessed and displayed by the computing device 530 and this information is used to initially permit access to the computing device 530. A subsequent scan of the physician ID tag or badge permits use of the workstation 500 by an authorized user through comparison, for example, with a list of authorized users that is stored into the memory of the computing device 530. It will be readily apparent, as noted previously, that other forms of encryption means that rely upon detection of a given biometric(s) can easily be utilized, such as, those shown, for example, in FIG. 21, to provide proper authorization and/or access to the diagnostic workstation 500 and to data which is stored therein.

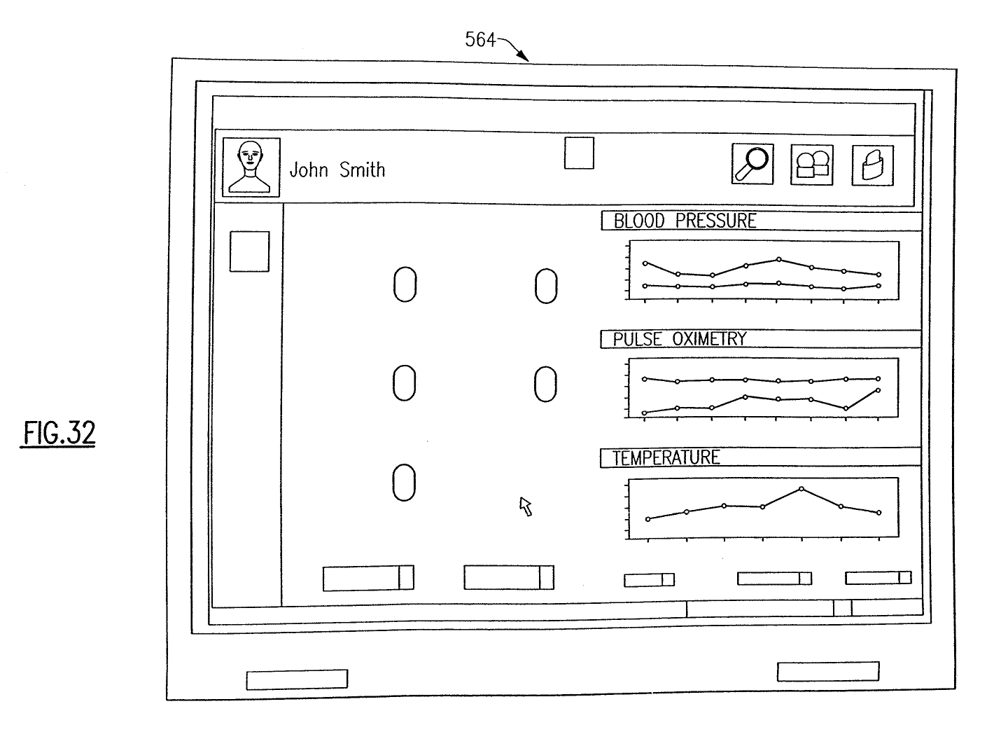

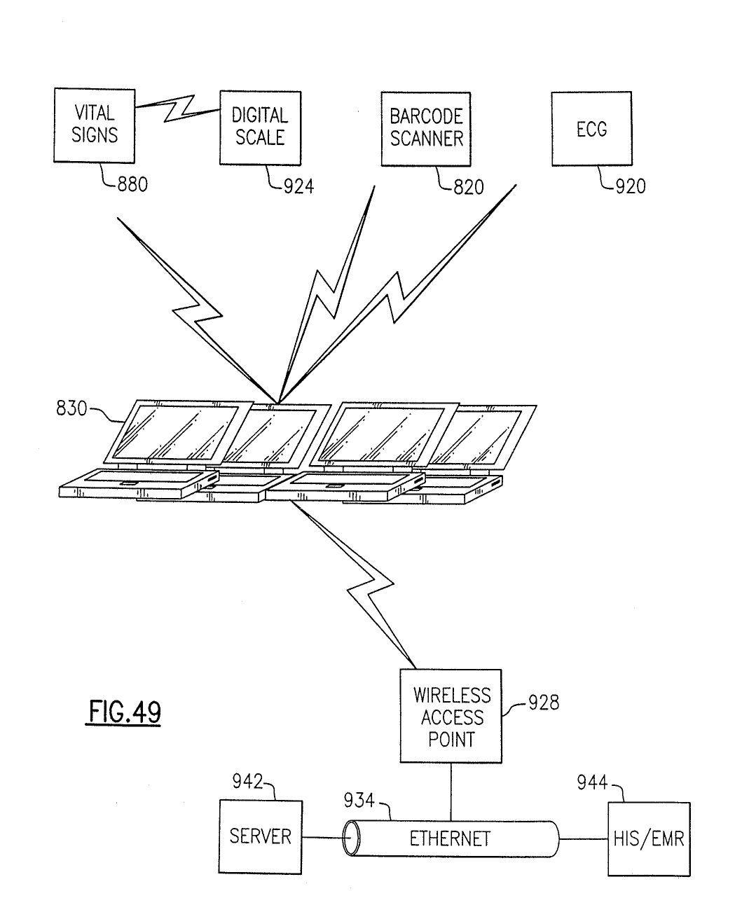

The entire operation of the workstation 500; that is, all of the diagnostic procedures that can be performed thereby, are preferably driven using the barcode scanner 540. As such, the collection of vital sign or other physiological parameter data, image data, and the like is preferably performed entirely using the scanner 540 and the set 550 of encoded instruction commands. In addition, the bar code scanner 540 can also track the use of patient-related items, such as disposable probe covers, tongue depressors, gloves, and the like as each are used by the physician or user, as well a summary of procedures that are performed for billing purposes. Each of the drawers 512 of the workstation can be designated for a specific patient, wherein each drawer can include not only spare probes or disposables, but medications as well. Medications can also be tracked using the present workstation 500 by scanning a med container having an appropriate bar code (not shown) using the scanner 540 in the same manner described above and subsequently transmitting the scanned data into the patient history as stored, along with the patient's vital sign readings and other information. By maintaining a history file for each patient, trending data can be realized. An example of an output display 564 having trending data is depicted in FIGS. 32 and 49. Another example is shown in FIG. 49.

In addition to image and other data, the scanner 540 can also capture digital signatures. Therefore, a barcode “box” can be created on paper with a signature space wherein the signature of a health care professional or the patient can be obtained and stored.

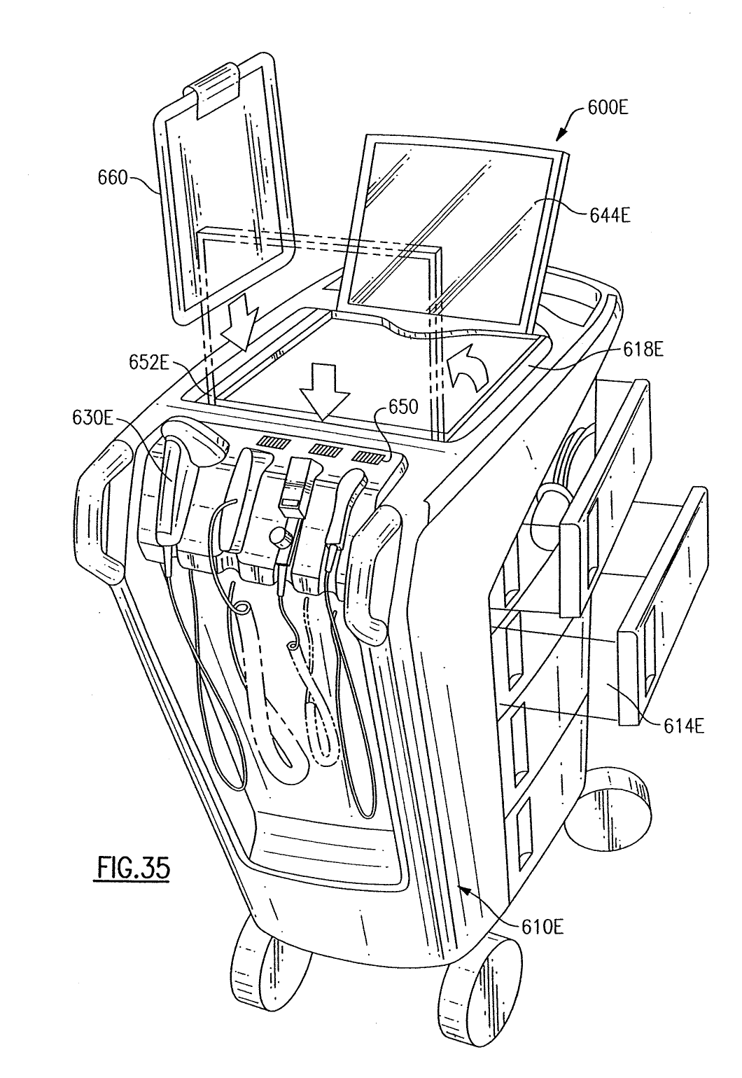

Using the above workstation 500 in this manner, errors are reduced and data compilation is much more comprehensive and complete. The computing device 530, upon detection of a proper bar code or other machine-readable symbol(s) from the instruction set 650 located on or near the workstation 500, as those for example shown in FIGS. 35-37, or separately, activates a stored macro or other software provided within the computing device 530 which produces the patient information to be displayed on display 534 as well as certain other executable commands such as image capture, print summary data sheet, review image data, show vital sign readings, etc. As shown in FIG. 30, a printer 560 can be disposed in one of the drawers 512 or can be connected thereto by other means and connected to the computing device 530 in a conventional manner to permit summary data sheets, such as those depicted in FIG. 32 to be outputted. The printer 560 can be physically connected or connected by a local area network (LAN) or alternatively by means of a wireless connection. The printer also can be located remotely from the workstation 500 in the latter instance.

The diagnostic workstation 500 can also be programmed to take patient vital signs on a predetermined schedule. Therefore, each workstation 500 can be configured to a specific patient's characteristics by data logging. That is, the workstation 500 can be programmed to take patient readings every fifteen minutes (or some other predetermined time period), with the readings being compared to existing or previous stored readings. An alert will be sounded if the readings, for example, drop by 20 percent or other predetermined value. In this manner, patients with higher than normal blood pressure readings can be compensated for as opposed to “normal” patients. These results can be examined for trends, such as those in FIG. 32. Moreover, these readings can be used to preset the capture of blood pressure readings automatically by comparing stored readings and by using the readings (e.g. a patient being hypo or hypertensive) to control the inflation of the blood pressure sleeve. Blood pressure sleeves can also be identified in advance of use by the workstation 500 through use, for example, of bar-coded or other machine-readable information labels that are disposed on the sleeve. These tags can include not only inventory information, but also can include, for example, the width of the sleeve, the information being scanned by the scanner 540 prior to use of the sphygmomanometer. This information can also be used to control the proper inflation of the sleeve automatically.

Though not shown, the cart 504 can also be equipped with a wireless transceiver that is interconnected to the computing device 530. As the cart 504 is wheeled through the clinic or hospital setting and past discrete wireless access points disposed throughout, data can be automatically retrieved and transmitted. This information can be transmitted to a secure server and be subsequently transmitted out, for example, via an Internet connection. Additionally, a microphone provided on the cart 504 and connected to the computing device permits audio data related to a patient encounter to be selectively captured and stored. This data can be archived with the other patient-related information and this audio data can also be transmitted in the form of .wav files by way of the Internet connection. Moreover, the workstation 500 can also include a real-time audio link through a network, as described in greater detail in a later embodiment, such as a hospital network, for consultation and emergency situations, or for requesting additional hardware, for example, if a device is not present in the patient's examination room.

### SIXTH AND ALTERNATIVE EMBODIMENTS

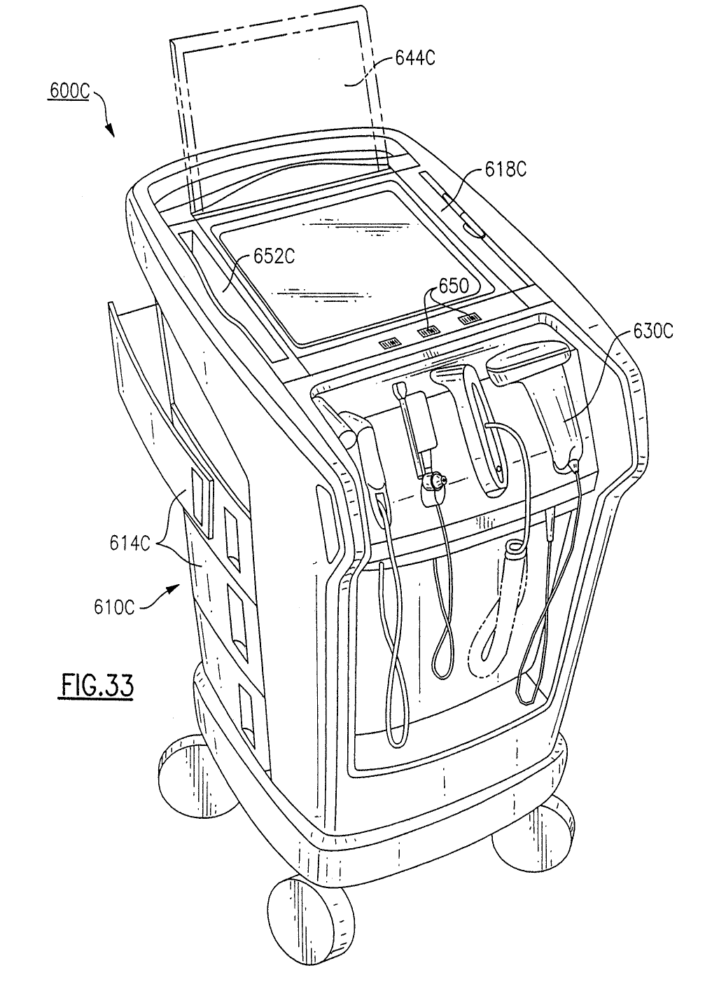

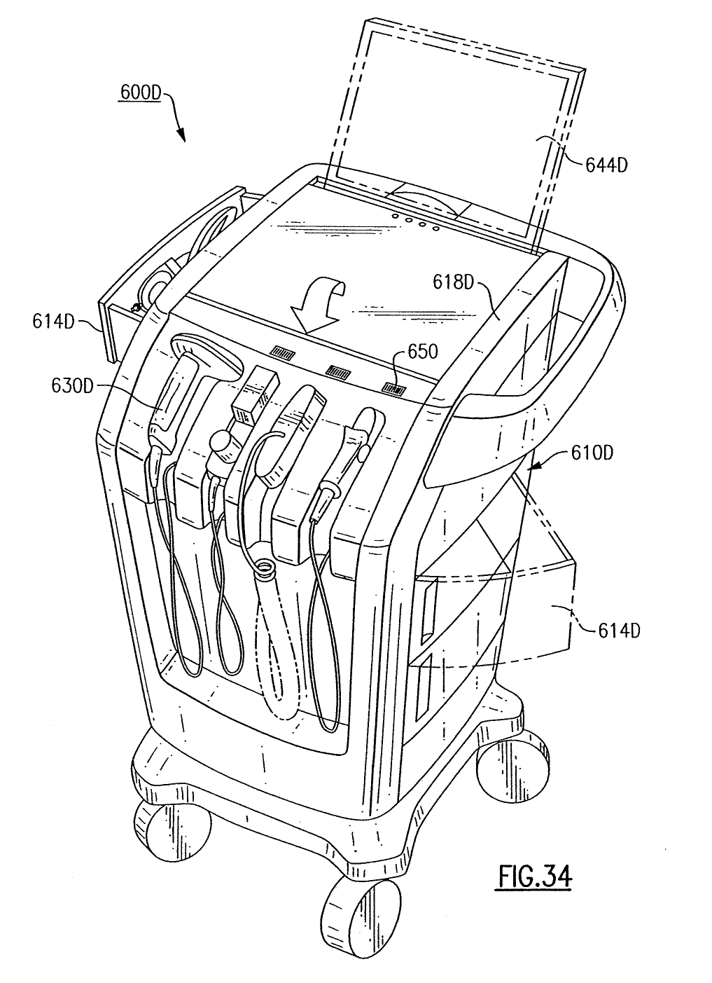

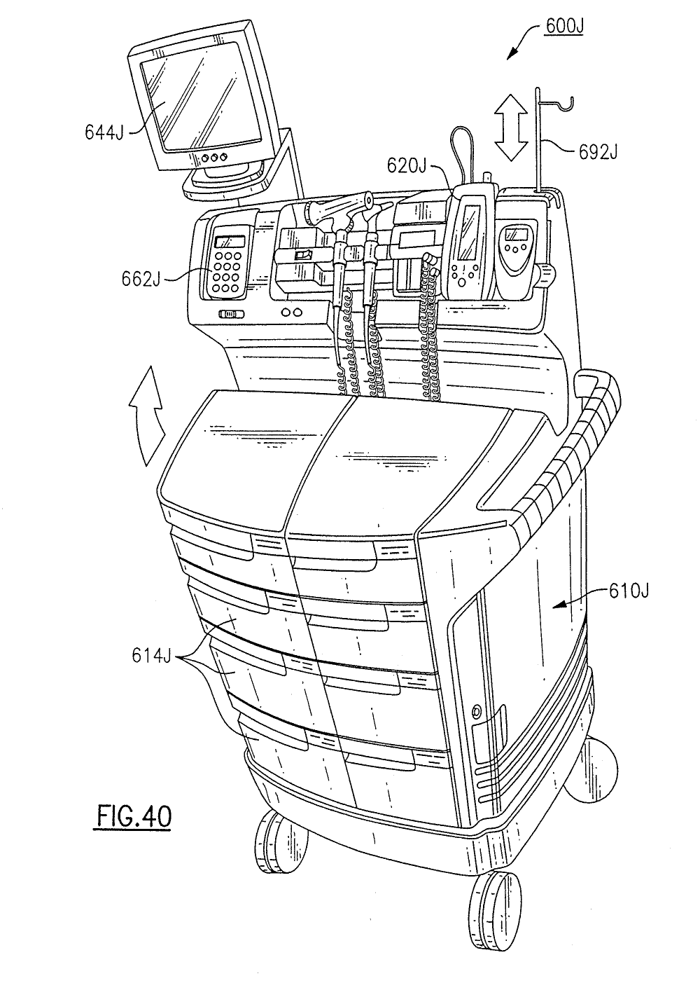

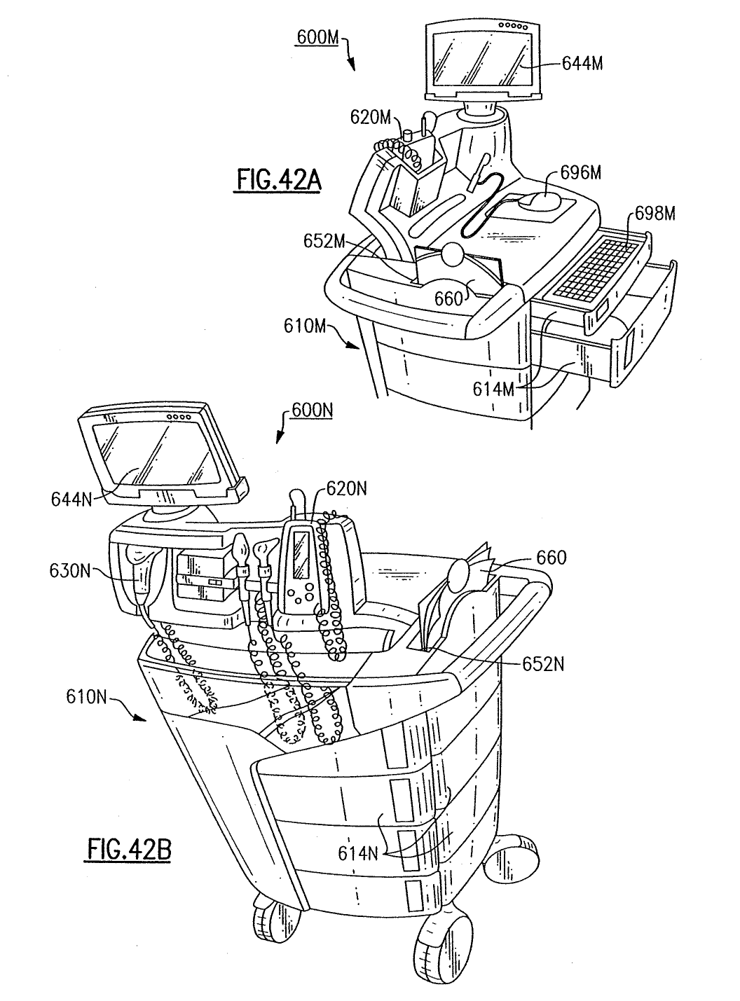

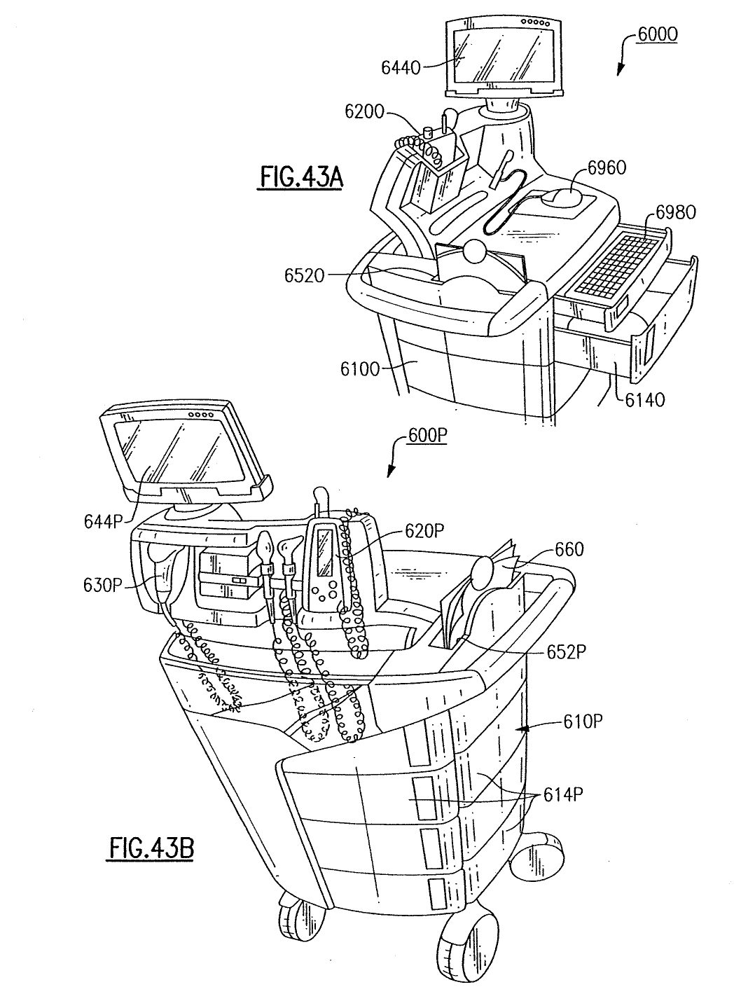

Referring in general to FIGS. 33-43, a medical diagnostic workstation 600 in accordance with multiple alternative embodiments of the invention also includes a movable cart 610 which defines cabinet-like supporting structure that further includes a plurality of drawers 614 on either lateral and front sides thereof that can be used for storage of items (blood pressure cuffs of varying size, spare probes, probe covers, medications, etc.) not integrated by the cart. A vital signs collector 620, such as the afore mentioned SPOT™ vital signs monitor manufactured by Welch Allyn, Inc., is arranged on a top supporting surface of each of the carts 610. These carts 610 each further include separate receiving receptacles arranged on a front side thereof for retaining a bar code scanner 630, such as the afore-mentioned IT-4410 HHP 2D-CCD scanner, as well as several hand-held diagnostic probe assemblies used by the vital sign collector 620, such as blood pressure, pulse oximetry, and body temperature probes. A computing device 640, in this instance a portable laptop computer, having a keyboard (not shown) and a display 644, is also housed at the top of each cart 610.

Each of these workstations 600 similarly integrate the vital signs collector 620 (and probe assemblies used therewith) with the computing device 640 and display with the bar code scanner 630 providing the means for primarily controlling the overall operation of the workstation using a set of instruction symbols (not shown in this embodiment) in the manner previously described.

As such, the bar code scanner 630 can control the taking of physiological parameter readings (e.g., vitals), transmission of parameter readings into the memory of the computing device 640, the display of patient related information, selective capture of images, and other operational features relating to patient/physician clinical encounters. In addition, any or all of this information can be outputted in a summary form, using a printer.

Each workstation 600 can also be used to accurately track inventory, medications and/or procedures, thereby providing a means for better reducing errors in billing and more effectively utilizing resources in the hospital setting.

As shown in each of FIGS. 33-43, alternative cart-like. diagnostic stations 600C-600P are illustrated, including a number of potential design variations for as noted above, illustrative purposes. Each of these movable workstations are capable of supporting a number of discrete components on a mobile cart structure 610C-610P, respectively, and commonly include a number of hand-held medical diagnostic instruments, a computing device 640C-640P, and an input device, such as a bar-code scanner 630C-630P that are retained by the cart.

In addition, each of the carts 610C-610P include a plurality of drawers 614C-614P for storage of disposables, medication, spare probes, or other items that could be useful in a patient encounter.

More particularly, the carts 610C, 610D and 610E of FIGS. 33-35, respectively each include all of the diagnostic instruments and the bar-code scanners 630C, 630D and 630E being housed in receptacles on a front facing surface of the cart. A set of instructions 650 written in machine-readable language are provided above the receptacles on the front facing surface or can be otherwise provided for convenience to the user.

The top surface 618C, 618D, 618E of each cart 610C, 610D, 610E houses the computing device 640C, 640D, 640E with the display 644C, 644D, 644E being capable of opening for use as shown. The top surfaces of carts 618C and 618E also include an adjacent vertical slot 652C, 652E, respectively, sized for retaining a patient chart 660, shown in FIG. 35.

FIG. 34 illustrates that at least one lateral drawer 614D can be opened pivotally for example, either toward or away from a patient bed.

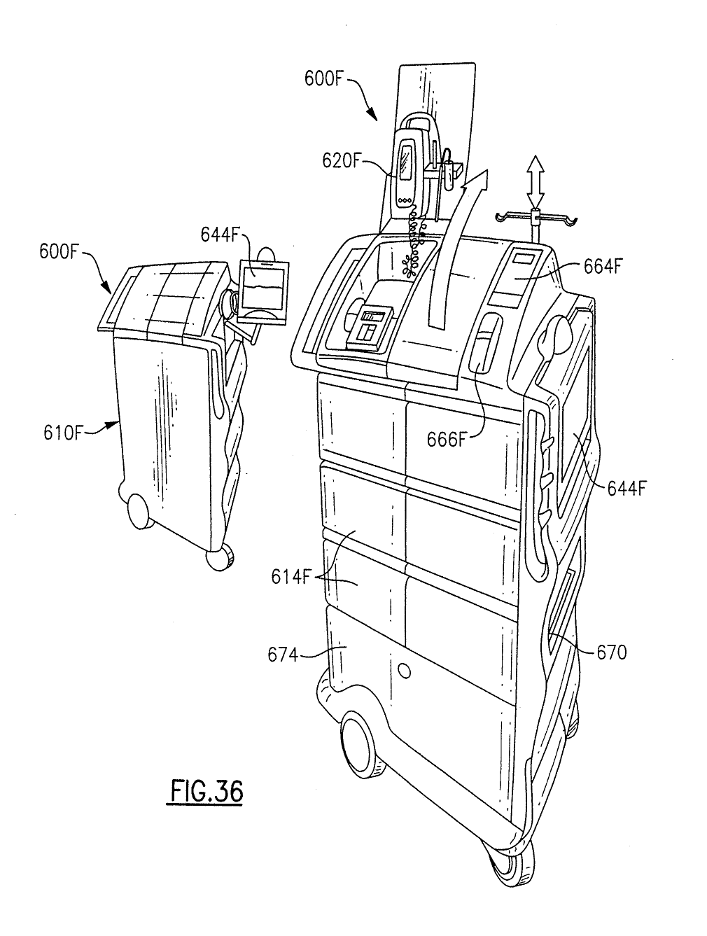

The workstation version 600F of FIG. 36 also illustrates a movable cart 610F with a plurality of drawers 614F. In this version, the medical instrument area that includes a vital signs collector 620F as well as a PDA 664 (Personal Data Assistant) such as a Palm® device and a telephone 666 each housed in separate receptacles. This version 600F further includes a video monitor 644F as a display that is hingably attached to one side of the cart housing. A printer (not shown) interior to the cart housing 610F includes an output tray 670 wherein the power supply and power distribution components are retained within a lower compartment 674 of the cart 610F.

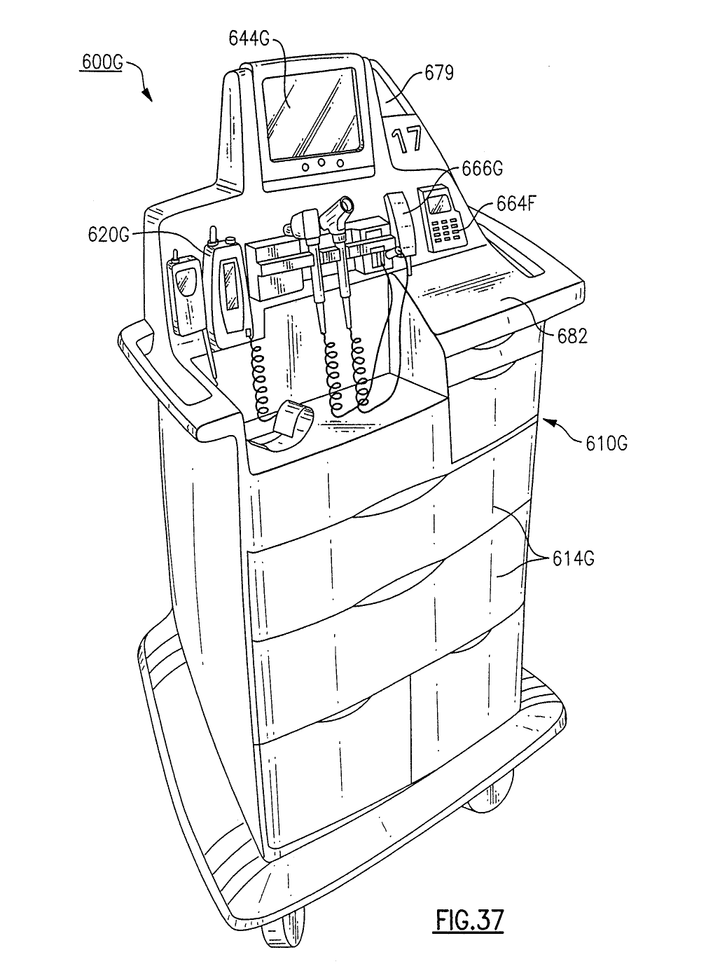

FIG. 37 illustrates a workstation design 600G similar to that described in FIG. 36 including each of the preceding components including a plurality of hand-held medical diagnostic instruments, a PDA 664G, a monitor 644G, a telephone 666G and a vital signs collector 620G. The workstation further includes an internal printer as well as a wireless link, such as an IRdA link 679 for permitting output to either a remote station or alternately for receiving input from another device. The workstation 600G also includes a working surface 682.

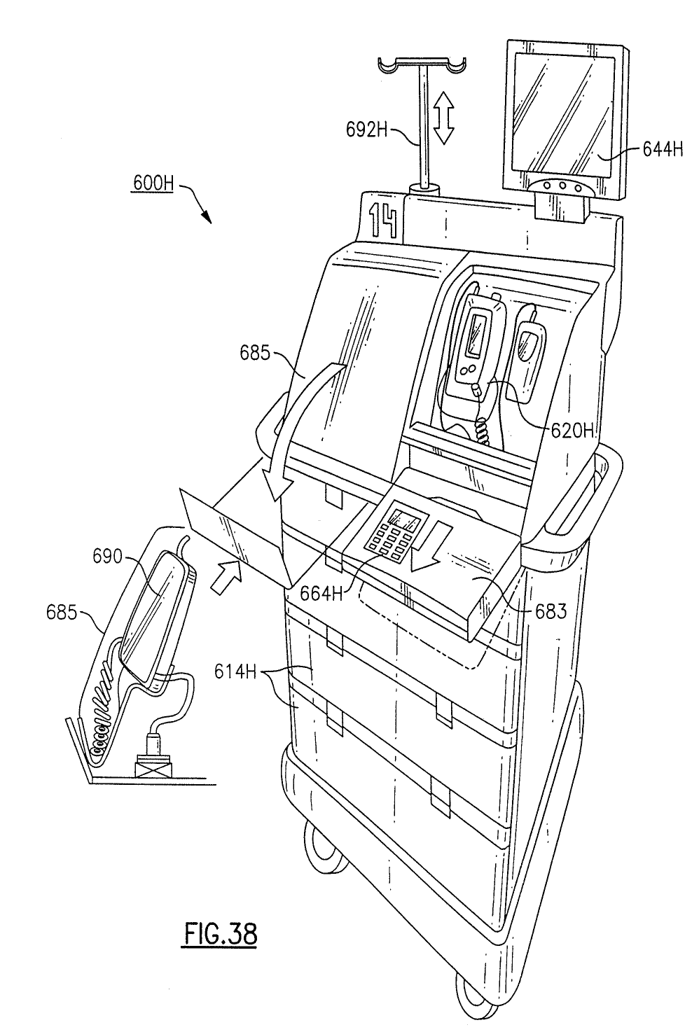

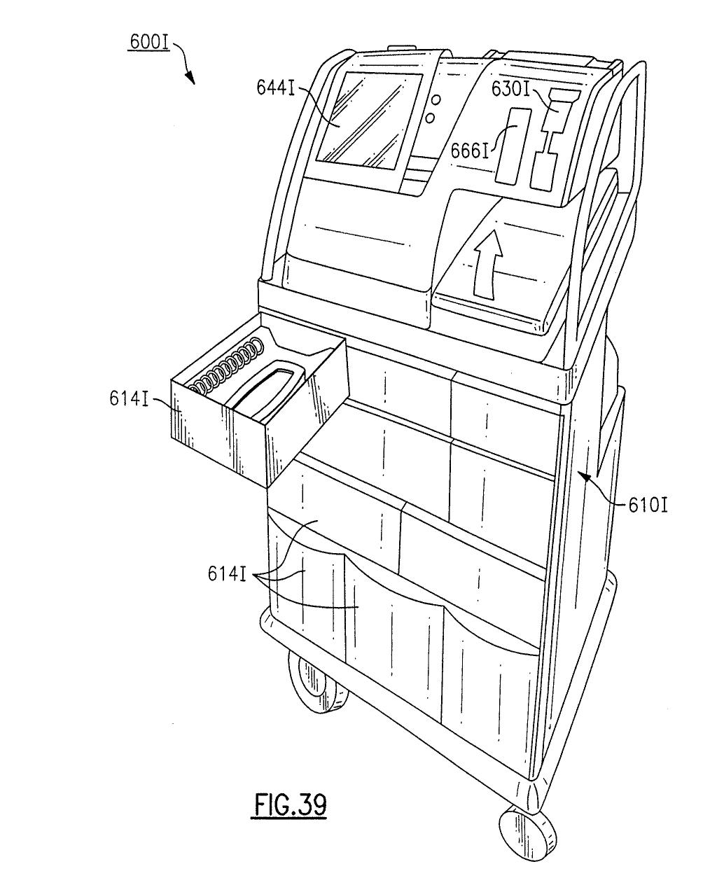

The workstation 600H of FIG. 38 illustrates a number of the preceding features as well as a flip-down work surface 683 that retains a PDA 664G as well as an adjacent front compartment 685 used to retain a medical instrument 690 such as a vital signs collector or other device. The cart 610H also includes a retractable IV pole 692 at the top thereof.

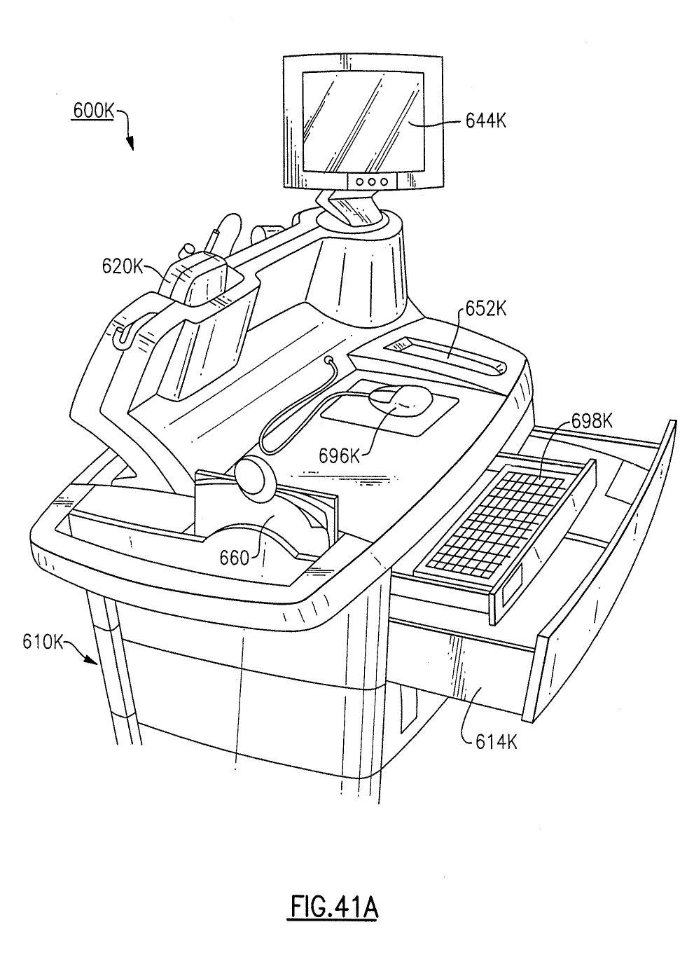

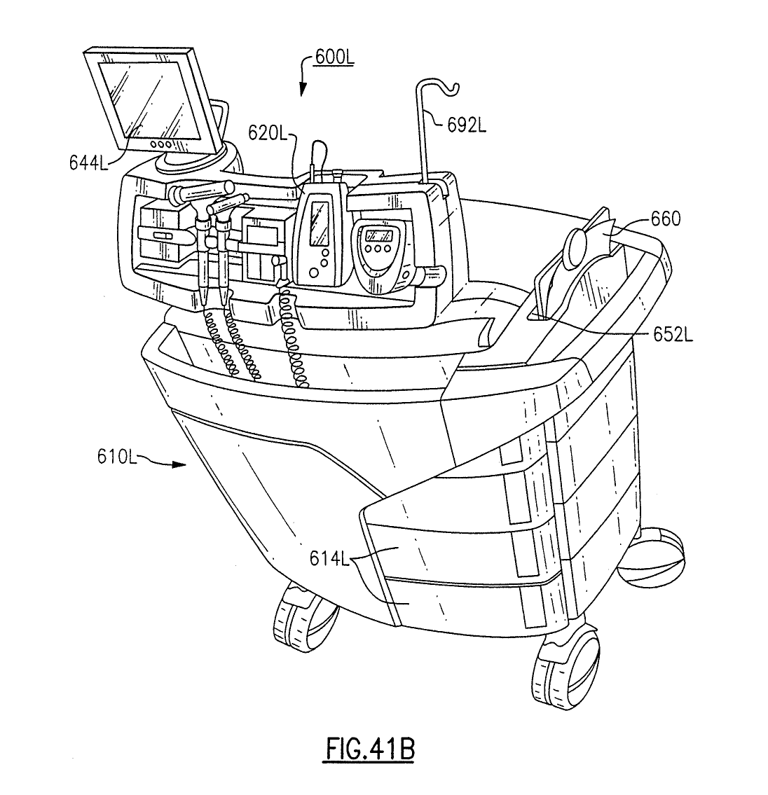

FIGS. 39-43 illustrate similar cart designs of the preceding. In addition, various interfaces as discussed infra can be introduced, such as keyboards shown as 698K, 698N and 6980 in FIGS. 41A, 42A and 43A, respectively, and/or mouse interfaces, shown as 696K, 696M and 6960 that can be used either in conjunction to control via the input device 630 or in lieu thereof. As noted in these figures, the number of varied cart designs is nearly limitless with assorted storage features for either equipment, probes, disposables, power supplies or the like and various storage locations on the console permitting either portable computing devices in the form of laptops and/or PDAs or tablet PC's to be used with the workstation.

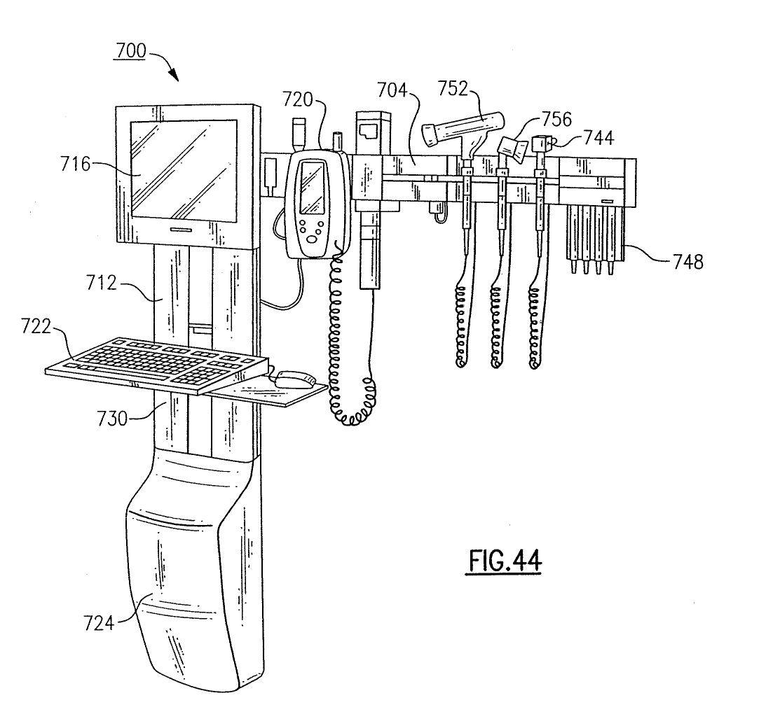

Each of the preceding embodiments, with the exception of the miniature video camera system disclosed in FIGS. 18 and 19, refer to a workstation defined by a movable supporting structure. It should be noted, however, that “fixed” versions are also clearly contemplated within the inventive concepts of this disclosure. For example, and referring to FIG. 44, a wall-mounted version of a medical diagnostic workstation 700 is herein shown and described.

The entirety of the herein described workstation 700 is attached to the wall of an examination room or similar setting that includes a wall support frame 704 for retaining a number of discrete components, including the following: a vital signs collector 720, such as a SPOT™ vital signs monitor, which as noted previously is capable of measuring blood pressure, saturation blood volume, pulse rate, and body temperature of a patient, a portable computing device 730, such as a portable laptop computer, and a plurality of diagnostic instruments. The wall support frame 704 contains a wall transformer as well as a plate that supports a vertical lift assembly 712 that retains an LCD monitor 716 and a keyboard 722. The workstation 700 further includes an enclosure 724 that retains power supplies and electrical connections and cabling for the components as well as other electrical equipment.

The above workstation 700 can be mounted to the wall of any examination room in a physician's office, clinic, hospital room, or other suitable area. The vital signs collector 720 is preferably releasably attached to the wall support 704 thereby permitting the collector, which includes its own power supply, to be used independently, if needed. The plurality of diagnostic instruments according to this embodiment includes an otoscope/throat illuminator 744, a specula dispenser 748, an ophthalmoscope 752, skin surface microscope 756, digital scale and/or other suitable apparatus. For example and though not shown other apparatus such as a colposcope and/or an otoscope and a rhinoscope could also be utilized herewith.

In a use case scenario, the above-described workstation 700 can be located in a central triage station. In this scenario, a nurse or clinical user brings the patient to the station wherein the patient's name can be typed into the workstation 700 using the keyboard 722 wherein the patient's history would already have been stored (if a current and not a “new” patient) into the memory of the computing device 730 for access.

Vital signs (e.g., SPO2, blood pressure, body temperature, pulse rate, etc.) can then be taken using the probe assemblies of the attached collector wherein the vital signs collector 720 is optically or otherwise wirelessly linked to the processor of the computing device 730 for capture, such as through a command that is typed into the keyboard 722. Once all of the vital signs have been taken, the user can selectively print the results onto a summary sheet (not shown), which preferably includes other patient information. This sheet can be attached, for example, to the patient's chart. The user can then bring the patient and his/her chart to the examination room. The physician who will examine the patient can then either review the patient chart or view this information on a mobile computing device (such as a PDA or laptop) before seeing the patient. The physician can then enter the examination room to perform, for example, a physical examination. During this exam, the physician can update the patient's chart by either writing onto the chart or by entering new information using the mobile computing device.

According to this embodiment, the computing device 730 and keyboard and display 716 are disposed within an adjustable assembly 712 that can be selectively positioned in terms of height, depending, for example, on whether the user is sitting of standing. In addition and as note previously, the vital signs collector 720 is preferably releasably attached to the wall support frame 704 to permit the collector to be used on its own, as needed.

Variations of the above system are evident, such as, for example, including either the bar-code scanner or the imaging device of the previous embodiments in the herein described wall mounted version.

### SEVENTH EMBODIMENT

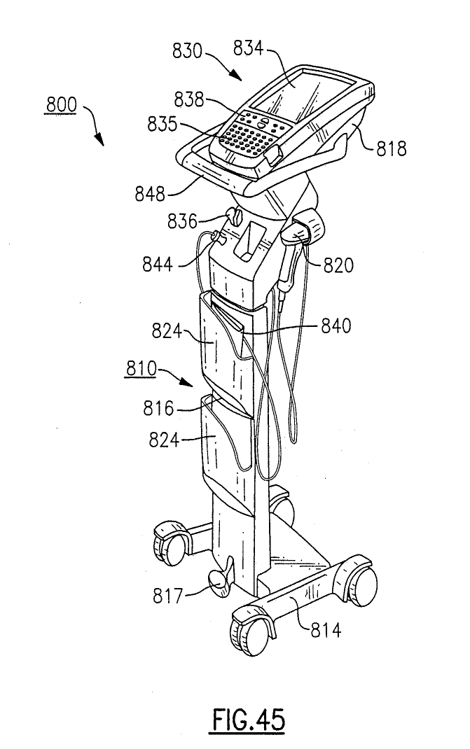

A seventh embodiment is herein described with reference to FIGS. 45-50. Referring first to FIG. 45, a mobile diagnostic workstation 800 includes a supporting structure 810 that supports a varied number of components, the components including an input device in the form of a bar code scanner 820, a computing device 830, a pulse oximeter 836, a sphygmomanometer 840 and an oral thermometer 844.

Like many of the embodiments that have been previously described above, the components are attached to the supporting structure 810 of the workstation 800. The workstation 800 is mobile, wherein the supporting structure 810 includes a wheeled base 814 having a foot brake 817. A vertical support post 816 of the supporting structure 810 includes a pair of pockets or storage receptacles 824 that can be used to store patient records, the sphygmomanometer 840, or alternatively patient disposables and/or medications.

The computing device 830 is supported in an upper portion of the supporting structure 810 and more particularly within a cavity defined within an angled top surface or console 818, permitting the display 834 of the computing device to be visible to the user. A keyboard 835 permits instructions to be inputted to the workstation 800 in addition to the operation being performed substantially by the bar code scanner 820, the scanner including an integrated miniature imager, the scanner being an IT 4410 2D CCD imaging scanner manufactured by Hand Held Products, Inc., though other suitable CCD and laser scanning devices could easily be substituted. In addition, a plurality of selective control buttons 838 are also disposed on the console 818, providing additional control options, as described in greater detail below, for a number of specific predetermined operations of the diagnostic workstation 800. A gripping handle 848 adjacent the upper portion of the vertical supporting post 818 permits the workstation 800 to be easily moved between patient rooms, as needed.

Prior to describing the overall operation of the workstation 800, discussion is made concerning its potential connectivity in a hospital or physician office environment.

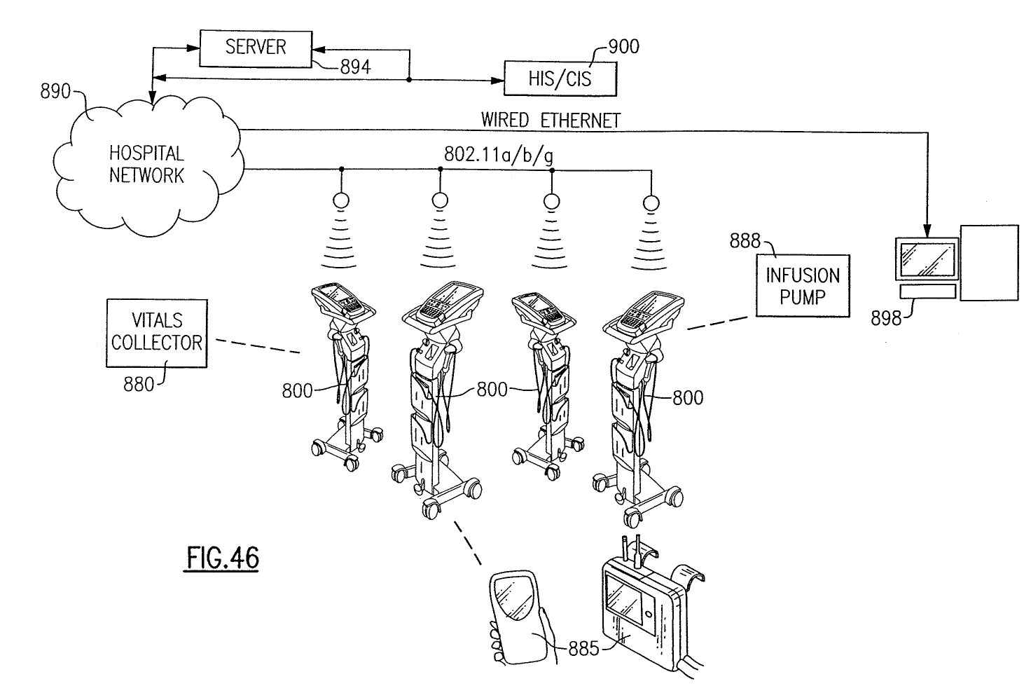

As shown in FIG. 46, a pictorial representation is made of the diagnostic workstation 800, by way of example, in connectivity with a plurality of physiological parameter measuring instruments and a hospital network. As shown therein, a number of workstations 800 are shown, mainly to show versatility, though a single workstation could also be so configured. In this embodiment, the workstation 800 is as defined in the foregoing, including the supporting structure 810 and the integrated computing device 830 and bar code scanner 820, as well as the pulse oximeter 836, sphygmomanometer 840 and oral thermometer 844. Each of the above components are hardwired in this embodiment to one another forming the integrated structure.

Still referring to FIG. 46, the workstation(s) 800 can be placed into wireless communication linkage using Bluetooth, WiFi or other wireless protocol with other components, and particularly with other devices in the patient room, for example a vital signs collector 880, such as the Spot Ultra vital signs collector manufactured by Welch Allyn, Inc., and an infusion pump 888, such as, for example those manufactured by Abbott Laboratories, Inc. The details of the particular protocol are known in the field and of themselves are not considered part of the invention. Similar connections can also be made between the workstation 800 and other portable devices 890, 894, such as other vital sign monitors such as the Welch Allyn Propaq and Welch Allyn Micropaq monitors.

The workstations 800 are further configured into a computer network 890 wherein data from the workstations is transmitted by means of a 802.lla/b/g protocol using a workstation server 894 that is further linked by an Ethernet connection to a remote computer review station 898 and a Computer Information System or Health Information System (CIS/HIS) 900, such as an Electronic Medical Record (EMR) system. In operation, the wireless connection between the instruments 880, 888 and the workstation 800 permits patient data to be acquired using the scanner or keyboard controls, or alternately a specific control button on the console 818 of the workstation. If the scanning device 830 is used, the patient ID is first scanned as well as that of the badge of the user to enable access of the workstation 800, as previously described. If the user is an authorized user for the system, then the scanning device 830 can be used in conjunction with machine-readable language instructions provided either on the display or otherwise on the workstation to initiate operations relating to the capture and storage of patient data and uploading of same to the hospital network 890. Specific controls such as the buttons provided on the console 818 can be used to control certain operations in lieu of the scanning device 830 or in conjunction therewith, for example, automatic blood pressure measurement wherein a sleeve can first be scanned by the scanning device to determine the width of the sleeve through machine readable information contained thereon, or alternately through manual keyboard entry. A pressure control system attached to the sleeve and connected to the computing device 830 then determines whether the patient has any predetermined blood pressure readings stored in memory and if so, then determines whether the patient is hypotensive or hypertensive so as to control the overall initial inflation of the sleeve.

As to the wireless control of each of the infusion pump 888 and the vital signs collector 880, the communications linkage with the workstation(s) 800 enables control of each so as to provide a virtual control interface at the workstation 800. Readings are taken, in the case of the vital signs collector 880 and are transmitted to the workstation 800. The readings are stored into memory of the computing device 830 and can then be uploaded onto the hospital network 890 either automatically when the workstation 800 passes an appropriate wireless access point in the hospital, or selectively by way of a control button 838 or by keyboard control enabling same.

Preferably, the workstation 800 can further include a microphone and speaker to enable audio messages to be made selectively by the user during a clinical encounter, as noted previously. The messages can be used, for example, prior to the onset of a new shift with regard to a patient and would replace the creation of written notes wherein the audio notes would be stored in conjunction with the remainder of the patient data. In addition, the wireless interconnection of the workstations 800 with the hospital network can include an additional communications linkage such as voice-over-IP or a cellular telephone link between a central nurse's station or other designated locations and the workstation(s).

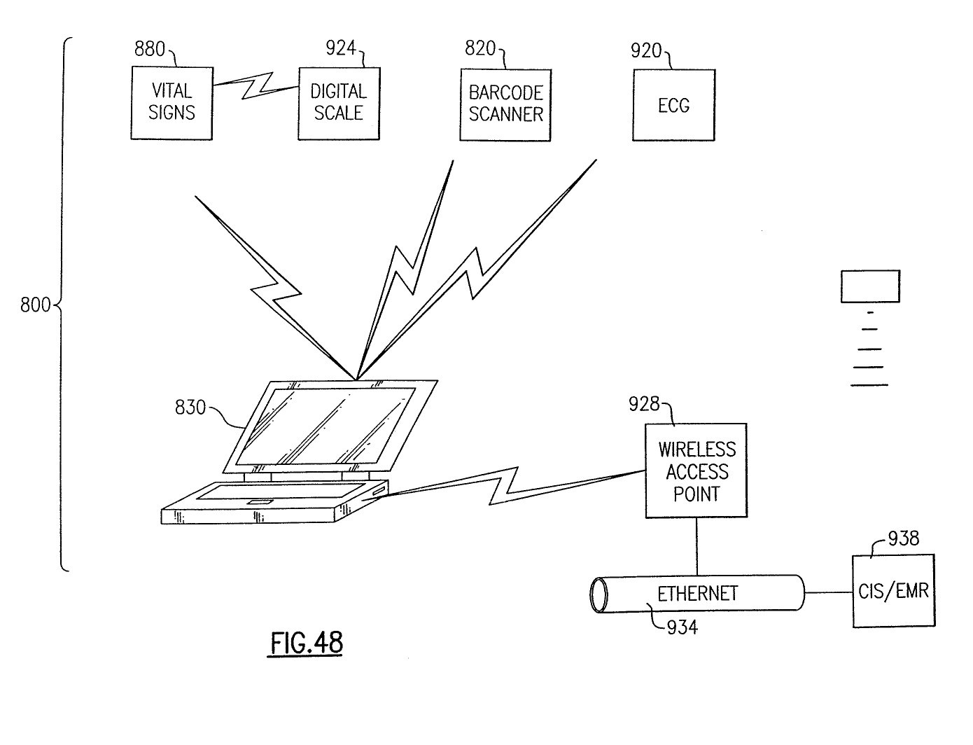

For purposes of the capability of the workstation 800 and in addition to the above diagnostic instruments, for purposes of the following figures, a 12-lead ECG assembly (shown diagrammatically in FIGS. 48-50) can also be serially or otherwise connected to the computing device 830 of the diagnostic workstation 800.

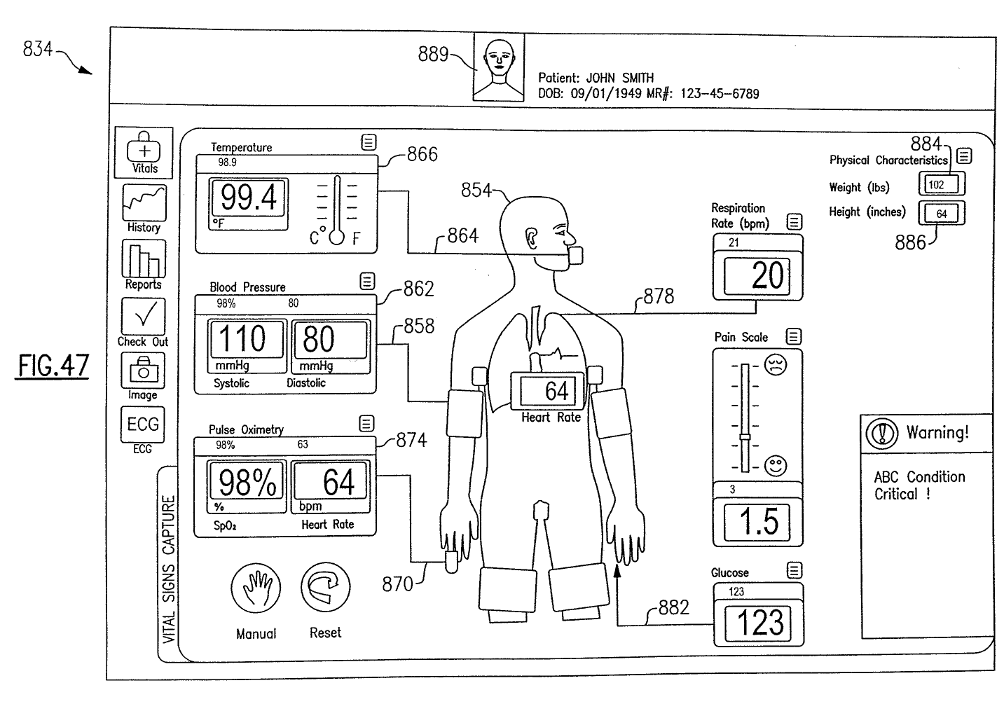

Referring to FIG. 47, there is shown an example of the display 834 of the workstation 800 in the form of a graphical user interface 850 that includes a format using a body image depiction 854 wherein at least one of the patient's physiological parameters being measured are represented in terms of the locale of the measurement that is being taken. For example, one such representation 858 is provided in the vicinity of the arm of the body image to indicate blood pressure which is displayed in a window 862. A second representation 864 is provided near the mouth to indicate body temperature indicated in a window 866, a third representation 870 being located near the finger of the body image to indicate pulse oximetry as shown by adjacent window 874. Similar representations and windows are provided for respiration 878 and glucose 882. Additionally, the patient's physical characteristics (weight, height) are displayed in windows 884, 886 and a digital image 889 of the patient is displayed at the top of the interface 850.

As shown on the left side of the display 834, additional features can be toggled by the user, the present depiction providing an overall representation of patient vitals. Additionally, a patient history of stored readings, of various reports and other devices, such as, for example ECG, can be accessed selectively by the user. near the lungs for respiration, the mouth for body temperature.

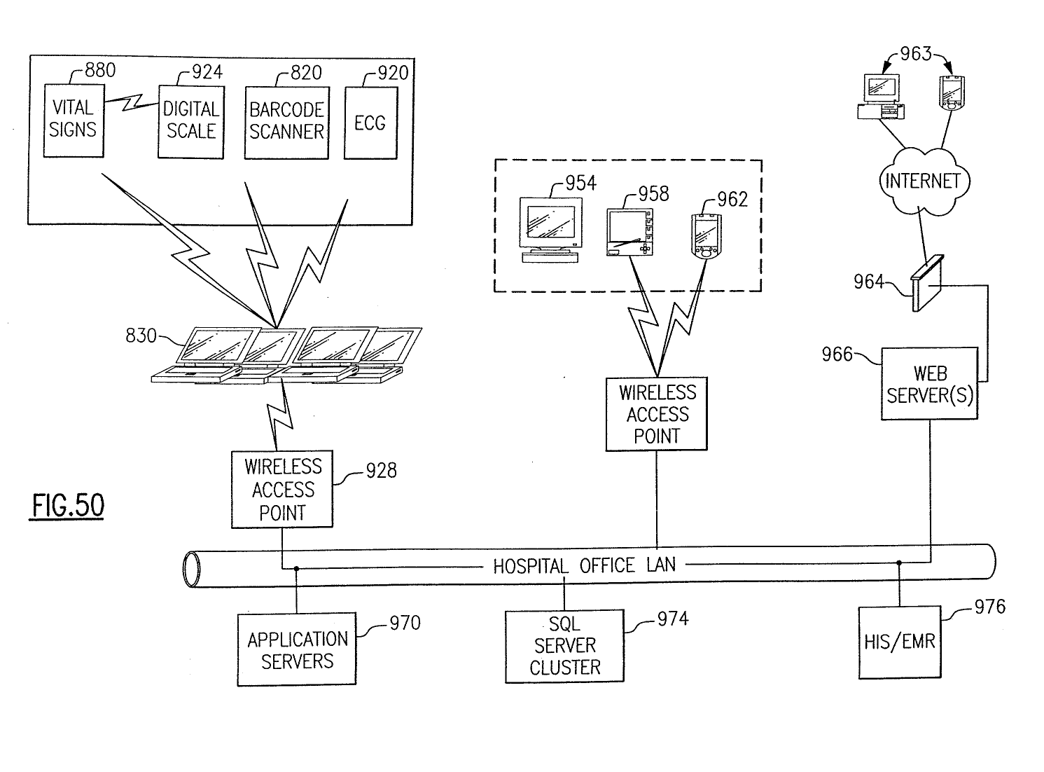

Referring to FIGS. 48-50, there are shown alternative embodiments for network connections involving the herein described diagnostic workstation 800. For purposes of this embodiment, certain specific integrated instruments have been selected for use with the workstation. In FIG. 50, a single workstation 800 is shown diagrammatically. According to this embodiment, the workstation 800 includes the integrated bar code scanner 820, a digital scale 924 made by Tanita, and an ECG assembly 920, such as Welch Allyn's Cardio Control Module, each of the foregoing components being hard wired, such as through a USB or other suitable connection, to the computing device 830 within the supporting structure (not shown) of the workstation. The vital signs collector 880 is wirelessly connected via Bluetooth or other suitable protocol thereto. In this embodiment as shown, the digital scale data is collected by the vital signs collector 880, with both sets of data being transmitted wirelessly to the workstation 800. Alternately, the digital scale 924 could communicate directly with the workstation 800.

The workstation 800, being mobile, is capable of uploading information when it passes an appropriate wireless access point 928 through connection with a hospital network, as previously noted, using an 802.11(b) or other suitable protocol in which the data can be transmitted to an CIS/EMR system 938 through an Ethernet connection 934.

Referring to FIG. 49, a small plurality of workstations (approximately 3-10 workstations) are shown for connection, each of the workstations also similarly including the wired connections with the scanning device 820, 12-lead ECG assembly 920 and the digital scale 924 with the computing device 830. Only one each of the above devices is shown for clarity purposes. The computing devices 830 are linked through an access point 928 (only one of which is shown) in the hospital setting to a server 942 and a Health Information System/Electronic Medical Record (HIS/EMR) system 944, the latter being suitably linked through a wired Ethernet connection 934.

Referring to FIG. 50, a larger plurality (greater than 20) of workstations 800 are depicted for use in a hospital/office local area network (LAN) 950 in which the computing devices 830 are linked wirelessly thereto by means of wireless access points 928. The network 950 further includes an interconnection whereby data from the workstation 800 can be uploaded to one of a computer workstation 954, a tablet PC 958, and/or a pocket PC 962, each of these components also being wirelessly linked to the network 950 by means of an 802.11 protocol. The network 950 also provides a remote Internet connection through a firewall 964 to a number of similar devices 963, the data being managed by an appropriate web server 966.

The network 950 also includes multiple servers in the form of application servers 970, an SQL(Structured Query Language) Server Cluster 974, and an HIS/EMR System 976, which allow for remote viewing and analysis of data collected by the workstation 800.

While the present invention has been particularly shown and described with reference to the preferred mode as illustrated in the drawings, it will be understood by one skilled in the art that various changes in detail may be effected therein without departing from the spirit and scope of the invention as defined by the claims.

Other features can be easily be added. For example, any of the preceding medical diagnostic workstations can include a global positioning system (GPS), such as those currently use in automobiles and other applications, as a means for tracking same using, for example, a central monitoring system in a hospital, clinic or other setting. According to another embodiment, the herein described diagnostic workstation can further include an RF interrogation device that can be used to track the location of other instruments or other articles that may be present in an examination room. The interrogation device transmits a radio frequency signal causing transmission of a return signal from a passive RF identification tag located on the instrument or article, thereby assisting in locating any particular instrument. The tag is extremely compact and is affixable, using for example thin film technologies, and includes device-specific information, such as the model number of the device and other pertinent data, as well as a miniature microprocessor having resident memory for storing the device specific information. Identification of the article is useful for saving time prior to conducting an examination, for example, if it is determined that the device being searched is already present in the examination room.

Specific diagnostics have been discussed throughout the course of discussion. It should be readily apparent however, that literally any form of testing can be performed using the herein described workstation. For example, ECG (12 lead), blood glucose, cholesterol, weight and drug delivery are other meaningful tests that can be performed using the above system, as well as multi-parameter monitoring. These tests can include, for example, immunoassay, molecular diagnostic, and proteomics analysis for disease states.

1. An integrated apparatus for use in a patient encounter, said apparatus comprising:
an input device having means for reading machine-readable information;
a computing device connected to at least one medical instrument and said input device; and
control means for controlling the operation of said at least one medical instrument, said input device, and said computing device wherein said apparatus is substantially and automatically controlled using said input device by scanning appropriate machine-readable information portions, said portions including instructions that are interpreted by and executed by said computing device to control operation of said apparatus.

2. An integrated apparatus according to claim 1, further including a miniature imaging device for selectively capturing images during a patient encounter.

3. An integrated apparatus according to claim 2, wherein said miniature imaging device is provided in said input device.

4. An integrated apparatus according to claim 1, wherein said input device is a bar-code scanner.

5. An integrated apparatus according to claim 1, including a printer connected to said computing device.

6. An integrated apparatus according to claim 1, wherein said control means further includes user-actuable controls for controlling the operation of the apparatus.

7. An integrated apparatus according to claim 2, wherein said computing device includes data storage means for selectively storing at least one image captured by said miniature imaging device and data from said at least one of said input device and said at least one medical instrument.

8. An integrated apparatus according to claim 2, wherein said input device includes means for identifying at least one machine readable portion in at least one image that is captured by said miniature imager device.

9. An integrated apparatus according to claim 8, wherein said computing device includes means for decoding said at least one machine readable portion, if said at least one portion is identified in a captured image.

10. An integrated apparatus according to claim 1, including encryption means for preventing unauthorized operation of said apparatus.

11. An integrated apparatus according to claim 1, wherein scanning of an appropriate machine-readable portion by said input device automatically causes activation of said at least one medical instrument.

12. An integrated apparatus according to claim 1, wherein at least one said machine-readable information portion is presented on a display of said apparatus.

13. An integrated apparatus according to claim 1, wherein at least one said machine-readable information portion is on a patient record sheet.

14. An integrated apparatus according to claim 13, wherein said patient record sheet further includes at least one machine-readable portion that includes patient-related data, wherein the patient-related data can be uploaded into the data storage means of said computing device and in which additional patient data can be added so as to create an updated patient record sheet having an updated machine readable portion thereon.

15. An integrated apparatus according to claim 1, wherein said input device and said computing device are each integrated onto a movable cart.

16. An integrated apparatus according to claim 2, wherein said miniature imaging device is wirelessly connected to said computing device.

17. An integrated apparatus according to claim 1, wherein said at least one medical instrument and said computing device are linked by means of at least one of a wireless protocol and a serial connection.

18. An integrated apparatus according to claim 1, including a power supply.

19. An integrated apparatus according to claim 18, wherein said power supply includes at least one rechargeable battery.

20. An integrated apparatus according to claim 1, wherein said at least one medical instrument is at least one of a medical vital signs monitor, a portable EKG assembly and a spyhygmomanometer.

21. An integrated apparatus according to claim 20, wherein said sphygmomanometer includes an inflatable sleeve having a pressure control assembly for inflating and deflating said sleeve, said pressure control assembly being connected to said computing device so as to inflate the sleeve to a predetermined pressure based on at least one previous blood pressure measurement of a specific patient whose blood pressure is being measured.

22. An integrated apparatus according to claim 21, wherein said computing device includes at least one database for storing patient physiological readings, said patient having means for identification via said input device, wherein said patient identification means automatically accesses said database and preprograms the pressure control assembly for said patient.

23. An integrated apparatus according to claim 1, wherein said control means includes at least a second input device for inputting instructions to said apparatus.

24. An integrated apparatus according to claim 23, wherein said second input device includes at least one of a keyboard and a mouse connected to said computing device.

25. An integrated apparatus according to claim 24, wherein said computing device includes data storage means and in which said second input device is a keyboard, said keyboard permitting manual entry of patient-related data into said data storage means.

26. An integrated apparatus according to claim 1, including means for training new users in the operation of said apparatus, said training means including a template that includes a plurality of machine-readable icons that are selectively openable by a user, wherein selection of an icon by said input device causes said computing device to open a portion of a training manual stored in the memory of said computing device.

27. An integrated apparatus according to claim 1, including inventory control means for tracking the use of disposable and nondisposable supply items relating to a patient.

28. An integrated apparatus according to claim 1, including means for tracking the delivery of medications to a patient.

29. An integrated apparatus according to claim 5, wherein said computing device can selectively produce a printable summary sheet containing at least one symbol having machine readable code thereupon, and in which patient related information is encoded in said at least one symbol to avoid redundancy in entering data and to permit updating.

30. An integrated apparatus according to claim 1, including means for determining the amount of fluid inputs and outputs of a patient.

31. An integrated apparatus according to claim 30, wherein at least one fluid container of a patient includes a plurality of machine readable indicators, each indicator being representative of a fluid level in said container, and in which said input device can selectively read at least one said indicator, said computing device having means for computing fluid amounts based on indicators that are read.

32. An integrated apparatus according to claim 26, wherein said display is attached to said movable cart in a manner that permits selective deployment of said display and storage when not in use.

33. An integrated apparatus according to claim 1, wherein said computing device includes at least one database for storing patient physiological readings, said patient having means for identification via said input device, wherein said patient identification means automatically accesses said database.

34. An integrated apparatus according to claim 7, wherein said data storage means includes archiving means for storing a history of patient physiological readings.

35. An integrated apparatus according to claim 34, wherein said computing device can selectively report results from said archiving means.

36. An integrated apparatus according to claim 1, wherein said apparatus is wall-mounted.

37. An integrated apparatus according to claim 12, wherein said display is a touch screen display.

38. An integrated apparatus according to claim 1, including means for interconnecting said apparatus to at least one network.

39. An integrated apparatus according to claim 1, wherein said network interconnecting means includes at least one wireless connecting means for wirelessly connecting said apparatus to at least a portion of said network.

40. An integrated apparatus according to claim 1, wherein at least one said medical instrument is integrated into said apparatus.

41. An integrated apparatus according to claim 1, including means for notifying a user when at least one selected physiological parameter monitored by said apparatus exceeds a predetermined threshold.

42. An integrated apparatus according to claim 1, including means for communicating with at least one medical device remote from said apparatus.

43. An integrated apparatus according to claim 42, wherein said at least one remote medical device is at least one of a vital signs monitor and an infusion pump.

44. An integrated apparatus according to claim 43, further including means for controlling the operation of said at least one remote medical device.

45. An integrated apparatus according to claim 42, wherein said remote communication means comprises wireless communication means, said wireless communication means permitting bi-directional communication between said at least one medical device and said apparatus.

46. An integrated apparatus according to claim 1, wherein said second input means includes means for entering manual measurements related to a patient.

47. An integrated apparatus according to claim 1, wherein said display includes a graphical user interface, said user interface including a body image format permitting a user to readily identify the patient physiological parameters being measured.

48. An integrated apparatus according to claim 47, wherein said body image format includes a scale body representation wherein physiological parameter readings of a patient are located in proximity to the actual location on the body that the parameter is being measured.

49. An integrated apparatus according to claim 1, wherein at least a portion of said control means are located on a keyboard.

50. An integrated apparatus according to claim 20, wherein said sphygmomanometer is automatically operated.

51. An integrated apparatus according to claim 1, wherein said at least one medical diagnostic instrument includes a weight scale.

52. An integrated apparatus according to claim 7, wherein said data storage means includes means for storing at least audio data added during said patient encounter.

53. An integrated apparatus according to claim 52, including means for transmitting said at least one audio message to a remote location.

54. An integrated apparatus according to claim 21, including means for determining the size of said blood pressure sleeve prior to inflation thereof.
